(19) United States
(12) Patent Application Publication
(10) Pub. No.: US 2023/0103260 A1
(43) Pub. Date: Mar. 30, 2023

(54) Genome Feature Extraction Method, Disease Prediction Method, Apparatus and Device

(71) Applicant: Alibaba (China) Co., Ltd., Hangzhou (CN)

(72) Inventors: Han Yang, Hangzhou (CN); Fei GU, Hangzhou (CN)

(21) Appl. No.: 17/725,354

(22) Filed: Apr. 20, 2022

(30) Foreign Application Priority Data
Apr. 30, 2021 (CN) 202110481497.3

(51) Int. Cl. G16B 30/00; G16B 40/20

(52) U.S. Cl. CPC G16B 30/00; G16B 40/20

(57) ABSTRACT

The present disclosures provide a genome feature extraction method, a disease prediction method, an apparatus and a device. The feature extraction method includes: obtaining a gene segment to be processed, the gene segment including a base quality; determining a confidence level corresponding to the gene segment based on the base quality; and performing a feature extraction operation on the gene segment based on the confidence level corresponding to the gene segment to obtain gene features of the gene segment. The embodiments effectively achieve an effective integration of the base quality into the gene features without increasing the dimensionality of data. In this way, not only the mode of implementation is simple and reliable and the completeness of extraction of gene features is ensured, but also the operation efficiency of an extraction operation on the gene features is improved.

### CROSS REFERENCE TO RELATED PATENT APPLICATIONS

This application claims priority to Chinese Patent Application No. 202110481497.3, filed on 30 Apr. 2021 and entitled “Genome Feature Extraction Method, Disease Prediction Method, Apparatus and Device,” which is hereby incorporated by reference in its entirety.

### TECHNICAL FIELD

The present disclosure relates to the field of gene detection, in particular to genome feature extraction methods, disease prediction methods, apparatuses and devices.

### BACKGROUND

Gene sequencing is a novel gene detection technology, which can analyze and determine a complete sequence of genes from blood or saliva, and predict the possibility of suffering from various types of diseases, and the behavior characteristics and behavior reasonableness of individuals. The gene sequencing technology can lock a personal lesion gene so as to perform precaution and treatment based on the personal lesion gene in advance.

A gene sequence is composed of a plurality of reads segments. Each read segment is a DNA segment with a specific length. The specific length depends on a reading length of a sequencer, and information in each read segment can include: a base sequence, a mass sequence, positive and negative chains, etc. The base sequence and the mass sequence correspond to each other one by one. For humans, a Reads segment covers 23 pairs of chromosomes, amounting to over 30 hundred million base pairs. Therefore, how to effectively detect mutation sites and related attributes of mutation points using massive sequencing information is a challenging task.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter. The term “techniques,” for instance, may refer to device(s), system(s), method(s) and/or processor-readable/computer readable instructions as permitted by the context above and throughout the present disclosure.

Embodiments of the present disclosure provide genome feature extraction methods, disease prediction methods, apparatuses and devices. Features of a gene segment are extracted through a confidence level of the gene segment. The mode of implementation is simple and reliable, and the completeness and efficiency of extracting features of a gene are effectively ensured.

In a first aspect, the embodiments of the present disclosure provide a genome feature extraction method, which includes:

obtaining a gene segment to be processed, the gene segment including a base quality;

determining a confidence level corresponding to the gene segment based on the base quality; and

performing a feature extraction operation on the gene segment based on the confidence level corresponding to the gene segment to obtain gene features of the gene segment.

In a second aspect, the embodiments of the present disclosure provide a genome feature extraction apparatus, which includes:

a first acquisition module configured to obtain a gene segment to be processed, the gene segment including a base quality;

a first determination module configured to determine a confidence level corresponding to the gene segment based on the base quality; and

a first extraction module configured to perform a feature extraction operation on the gene segment based on the confidence level corresponding to the gene segment to obtain gene features of the gene segment.

In a third aspect, the embodiments of the present disclosure provide an electronic device, includes: a memory and a processor, wherein the memory is configured to store one or more computer instructions, and wherein the one or more computer instructions, when executed by the processor, implement the genome feature extraction method according to the first aspect.

In a fourth aspect, the embodiments of the present disclosure provide a computer storage medium used for storing a computer program, which, when executed by a computer, implements the genome feature extraction method according to the first aspect.

In a fifth aspect, the embodiments of the present disclosure provide a disease prediction method, which includes:

obtaining a disease prediction request, the disease prediction request including a gene segment to be predicted;

determining gene features corresponding to the gene segment included in the disease prediction request, the gene features being extracted based on a confidence level of the gene segment, wherein the confidence level of the gene segment is related to a base quality included in the gene segment; and

performing disease prediction on the gene segment based on the gene features to obtain a disease prediction result.

In a sixth aspect, the embodiments of the present disclosure provide a disease prediction apparatus, which includes:

a second obtaining acquisition module configured to obtain a disease prediction request, the disease prediction request including a gene segment to be predicted;

a second determination module configured to determine gene features corresponding to the gene segment included in the disease prediction request, wherein the gene features are extracted based on a confidence level of the gene segment, and the confidence level of the gene segment is related to a base quality included in the gene segment; and

a second prediction module configured to perform disease prediction on the gene segment based on the gene features to obtain a disease prediction result.

In a seventh aspect, the embodiments of the present disclosure provide an electronic device, which includes: a memory and a processor, wherein the memory is configured to store one or more computer instructions, and wherein the one or more computer instructions, when executed by the processor, implement the disease prediction method according to the fifth aspect.

In an eighth aspect, the embodiments of the present disclosure provide a computer storage medium for storing a computer program, which, when executed by a computer, implements the disease prediction method according to the fifth aspect.

In a ninth aspect, the embodiments of the present disclosure provide a genome feature extraction method, which includes:

determining a processing resource corresponding to a feature extraction service of a genome in response to a feature extraction request for calling the genome; and

performing the following steps using the processing resource: obtaining a gene segment to be processed, the gene segment including a base quality; determining a confidence level corresponding to the gene segment based on the base quality; and performing a feature extraction operation on the gene segment based on the confidence degree corresponding to the gene segment to obtain gene features of the gene segment.

In a tenth aspect, the embodiments of the present disclosure provide a genome feature extraction apparatus, which includes:

a third determination module configured to determine a processing resource corresponding to a feature extraction service of a genome in response to a feature extraction request for calling the genome;

a third processing module configured to perform the following steps using the processing resource: obtaining a gene segment to be processed, the gene segment including a base quality; determining a confidence level corresponding to the gene segment based on the base quality; and performing a feature extraction operation on the gene segment based on the confidence degree corresponding to the gene segment to obtain gene features of the gene segment.

In an eleventh aspect, the embodiments of the present disclosure provide an electronic device, which includes: a memory and a processor, wherein the memory is configured to store one or more computer instructions, and wherein the one or more computer instructions, when executed by the processor, implement the genome feature extraction method according to the ninth aspect.

In a twelfth aspect, the embodiments of the present disclosure provide a computer storage medium for storing a computer program, which, when executed by a computer, implements the genome feature extraction method according to the ninth aspect.

In a thirteenth aspect, the embodiments of the present disclosure provide a disease prediction method, includes:

determining a processing resource corresponding to a disease prediction service in response to a call for a disease prediction request; and

performing the following steps using the processing resource: obtaining the disease prediction request, the disease prediction request including a gene segment to be predicted; determining gene features corresponding to the gene segment included in the disease prediction request, the gene features being extracted based on a confidence level of the gene segment, wherein the confidence level of the gene segment is related to a base quality included in the gene segment; and performing disease prediction on the gene segment based on the gene features to obtain a disease prediction result.

In a fourteenth aspect, the embodiments of the present disclosure provide a disease prediction apparatus, which includes:

a fourth determination module configured to determine a processing resource corresponding to a disease prediction service in response to a call for a disease prediction request;

a fourth processing module configured to perform the following steps using the processing resource: obtaining the disease prediction request, the disease prediction request including a gene segment to be predicted; determining gene features corresponding to the gene segment included in the disease prediction request, the gene features being extracted based on a confidence level of the gene segment, wherein the confidence level of the gene segment is related to a base quality included in the gene segment; and performing disease prediction on the gene segment based on the gene features to obtain a disease prediction result.

In a fifteenth aspect, the embodiments of the present disclosure provide an electronic device, including: a memory and a processor, wherein the memory is configured to store one or more computer instructions, and wherein the one or more computer instructions, when executed by the processor, implement the disease prediction method according to the thirteenth aspect.

In a sixteenth aspect, the embodiments of the present disclosure provide a computer storage medium for storing a computer program, which, when executed by a computer, implements the disease prediction method according to the thirteenth aspect.

In a seventeenth aspect, the embodiments of the present disclosure provide a genome feature extraction method, which includes:

displaying an interactive interface used for realizing a feature extraction operation of a genome;

obtaining a gene segment to be processed in response to an execution operation inputted to the interactive interface, the gene segment including a base quality;

determining gene features of the gene segment and a data acquisition quality corresponding to the gene features, wherein the gene features are obtained by extraction based on a confidence level of the gene segment, and the confidence level of the gene segment is related to the base quality; and

displaying the gene features and the data acquisition quality in the interactive interface.

In an eighteenth aspect, the embodiments of the present disclosure provide a genome feature extraction method, which includes:

a fifth display module configured to display an interactive interface used for realizing a feature extraction operation of a genome;

a fifth acquisition module configured to obtain a gene segment to be processed in response to an execution operation inputted to the interactive interface, the gene segment including a base quality;

a fifth processing module configured to determine gene features of the gene segment and a data acquisition quality corresponding to the gene features, wherein the gene features are obtained by extraction based on a confidence level of the gene segment, and the confidence level of the gene segment is related to the base quality; and

the fifth display module further configured to display the gene features and the data acquisition quality in the interactive interface.

In a nineteenth aspect, the embodiments of the present disclosure provides an electronic device, including: a memory and a processor, wherein the memory is configured to store one or more computer instructions, and wherein the one or more computer instructions, when executed by the processor, implement the genome feature extraction method according to the seventeenth aspect.

In a twentieth aspect, the embodiments of the present disclosure provide a computer storage medium for storing a computer program, which, when executed by a computer, implements the genome feature extraction method according to the seventeenth aspect.

According to the technical solutions provided by the embodiments, a gene segment to be processed is obtained, and a confidence level corresponding to the gene segment is then determined based on a base quality included in the gene segment. A feature extraction operation is performed on the gene segment based on the confidence level corresponding to the gene segment, to obtain gene features of the gene segment, which effectively achieve an effective integration of the base quality into the gene features without increasing the dimensionality of data. In this way, not only the mode of implementation is simple and reliable and the completeness of extraction of gene features is ensured, but also the operation efficiency of an extraction operation on the gene features is improved, thus further improving the practicability of the technical solutions.

### BRIEF DESCRIPTION OF THE DRAWINGS

In order to more clearly illustrate the technical solutions in the embodiments of the present disclosure or existing technologies, drawings used in the description of the embodiments or the existing technologies will be briefly described below. Apparently, the drawings in the following description represent some embodiments of the present disclosure, and one skilled in the art can also obtain other drawings according to these drawings without making any creative effort.

FIG. 1 is a schematic diagram of a scenario of a genome feature extraction method according to the embodiments of the present disclosure.

FIG. 2 is a schematic flowchart of a genome feature extraction method according to the embodiments of the present disclosure.

FIG. 3 is a schematic diagram of a process for obtaining a gene segment to be processed according to the embodiments of the present disclosure.

FIG. 4 is a schematic diagram of obtaining a gene segment to be processed according to the embodiments of the present disclosure.

FIG. 5 is a schematic diagram of mutation detection of a gene segment based on gene features according to the embodiments of the present disclosure.

FIG. 6 is a flow chart of a disease prediction method according to the embodiments of the present disclosure.

FIG. 7 is a schematic block diagram of a method for processing genome analysis according to the embodiments of the present disclosure.

FIG. 8 is a schematic diagram of a three-dimensional convolutional neural network for analyzing and processing gene features according to the embodiments of the present disclosure.

FIG. 9 is a schematic flowchart of a method for extracting features of a genome according to the embodiments of the present disclosure.

FIG. 10 is a schematic flowchart illustrating a disease prediction method according to the embodiments of the present disclosure.

FIG. 11 is a schematic structural diagram of a genome feature extraction apparatus according to the embodiments of the present disclosure.

FIG. 12 is a schematic structural diagram of an electronic device corresponding to the genome feature extraction apparatus provided in the embodiment shown in FIG. 11.

FIG. 13 is a schematic structural diagram of a disease prediction apparatus according to the embodiments of the present disclosure.

FIG. 14 is a schematic structural diagram of an electronic device corresponding to the disease prediction apparatus provided in the embodiment shown in FIG. 13.

FIG. 15 is a schematic structural diagram of another genome feature extraction apparatus according to the embodiments of the present disclosure.

FIG. 16 is a schematic structural diagram of an electronic device corresponding to the genome feature extraction apparatus provided in the embodiment shown in FIG. 15.

FIG. 17 is a schematic structural diagram of another disease prediction apparatus according to the embodiments of the present disclosure.

FIG. 18 is a schematic structural diagram of an electronic device corresponding to the disease prediction apparatus provided in the embodiment shown in FIG. 17.

FIG. 19 is a schematic diagram of another genome feature extraction method according to the embodiments of the present disclosure.

FIG. 20 is a schematic structural diagram of another genome feature extraction apparatus according to the embodiments of the present disclosure.

FIG. 21 is a schematic structural diagram of an electronic device corresponding to the genome feature extraction apparatus according to the embodiment shown in FIG. 20.

FIG. 22 is a schematic structural diagram of the example apparatuses described in FIGS. 11, 13, 15, 17, and 20 in further details.

### DETAILED DESCRIPTION

In order to make the objectives, technical solutions and advantages of the embodiments of the present disclosure clearer, the technical solutions in the embodiments of the present disclosure will be clearly and completely described below with reference to the drawings in the embodiments of the present disclosure. Apparently, the described embodiments represent some, but not all, of the embodiments of the present disclosure. All other embodiments, which can be derived by one skilled in the art from the embodiments given herein without making any creative effort, shall fall within the scope of protection of the present disclosure.

The terminology used in the embodiments of the present disclosure is intended to describe particular embodiments only, and is not intended to limit the present disclosure. As used in the examples of the present disclosure and the appended claims, singular forms “a”, “an”, and “the” are intended to include plural forms as well, and “a” and “an” generally include at least two, but do not exclude at least one, unless the context clearly dictates otherwise.

It should be understood that the term “and/or” as used herein is merely a type of association that describes associated objects, which means that three relationships may exist. For example, A and/or B may mean: three situations, namely, A exists alone, A and B exist simultaneously, and B exists alone. In addition, the character “/” herein generally indicates that the former and latter related objects are in an “or” relationship.

The word “if”, as used herein, may be interpreted as “at the time when . . . ” or “when . . . ” or “in response to determining” or “in response to detecting”, depending on the context. Similarly, the phrases “if determining” or “if detecting (a stated condition or event)” may be interpreted as “when determining” or “in response to determining” or “when detecting (a stated condition or event)” or “in response to detecting (a stated condition or event)”, depending on the context.

It is also noted that the terms “including”, “containing” or any other variation thereof, are intended to cover a non-exclusive inclusion, such that a product or system that includes a list of elements does not include only those elements, but may also include other elements that are not expressly listed or that are inherent to such product or system. Without further limitation, an element defined by the phrase “including a . . . ” does not exclude the presence of other identical elements in a product or system that includes such element.

In addition, a time sequence of steps in each method embodiment described below is only an example and is not strictly limited.

### Definition of Terms

Gene sequencing: is a novel gene detection technology, can analyze and determine the complete sequence of genes from blood or saliva, and can predict the possibility of suffering from various diseases and the behavior characteristics and behavior reasonableness of individuals. The gene sequencing technology can lock a personal lesion gene so as to perform precaution and treatment based on the personal lesion gene in advance.

Mutation analysis: genetic variation refers to a sudden heritable variation that occurs in a genomic DNA molecule. At the molecular level, genetic variation refers to a structural change in base pair composition or arrangement of genes. Although being relatively stable, genes are able to replicate themselves precisely at cell division. However, such stability is relative. Under some conditions, a gene can also be suddenly changed from its original form to a new form. In short, a new gene is suddenly appeared at a site to replace the original gene.

SNP: single nucleotide polymorphism refers to DNA sequence polymorphism caused by variation of a single nucleotide at the genomic level. It is the most common one of human heritable variations, accounting for over 90% of all known polymorphisms. SNP is widely present in the human genome, averaging 1 per 300 base pairs, and the total number is estimated to be 3 million or even more. A SNP is a two-state marker, caused by a transition or transversion of a single base, or by an insertion or deletion of a base. SNP may be in either a gene sequence or a non-coding sequence outside a gene.

Indel: insertion-deletion, which is as translated as an indel marker, refers to a difference between two parents in the entire genomes. One parent has a certain number of nucleotide insertions or deletions in its genome relative to the other parent. Based on insertion and deletion sites in the genome, primers for Polymerase Chain Reaction (PCR) for amplifying the insertion and deletion sites are designed, which are InDel markers.

Reads: refers to a piece of DNA of a specific length, which is determined by the reading length of a sequencer.

Deep learning: is to lean intrinsic rules and expression levels of sample data, and information obtained in these learning processes is very helpful for interpreting data such as characters, images and sounds, etc. The final goal thereof is to enable a machine to possess the analysis and learning capability like a human, and to recognize data such as characters, images and sounds, etc.

In order to understand specific processes of implementation of the technical solutions in this embodiment, related technologies are described as follows:

For humans, a Reads segment covers 23 pairs of chromosomes, amounting to more than 3 billion base pairs. Information in each read segment may include: base sequence(s), mass sequence(s), strand(s), etc., wherein the base sequence(s) and the mass sequence(s) correspond to each other one by one. For any one candidate location, there may be many reads covering such location. A candidate location is a small segment of 3 billion, and it is a relatively large task to extract complete and valid gene features from all the reads covering this location.

At present, there are two main ways to extract gene features:

(1) Performing feature extraction using a deep neural network (Deepvariant), with an image of a large-size pileup format being used in the process of feature extraction.

This method can convert a sequencing depth, a sequence range and an information type into the length, the width and the depth of an image (namely an image channel). Generally, the length and the width are both greater than 200, and the depth mainly includes: bases, a base quality, a mapping quality, positive and negative strands, etc. In a feature extraction mode using Deepvariant, extracted feature information is more complete, but the calculation of a large-size image brings a limit on the data processing speed. The time consumed by an inference process is 10 times longer than that of a Clair-based implementation method. Tens of hours are generally consumed in the process of analyzing a single human genome. Although the contained information is more complete, the operation efficiency seriously limits the usage scene of this method.

(2) Performing feature extraction using a linear model (Clair), with an image of a small-size pileup format being used in the process of feature extraction.

This method can integrate sparse information of all reads segments in a statistical manner. Specifically, all information can be stored in a three-dimensional array, and three dimensions respectively represent: position information (for example, data length as 33) centering around candidate positions, positive and negative strands (A, G, C, T, A−, G−, C−, T−) corresponding to four different bases, and four different types of statistical information (a statistic of bases being identical to reference bases, a statistic of base insertions, a statistic of base deletions, and a statistic of single nucleotide alternative bases).

The Clair-based feature extraction method needs a smaller number of calculations, is faster in speed, and has an operation efficiency that is more than 10 times faster than that of the implementation method using Deepvariant. However, the obtained feature information includes bases, a mapping quality, and positive and negative strand information, while base quality information is completely ignored. Since the base quality describes the reliability of a base detected at each site, it is very important to the analysis of gene sequencing data. As can be seen, the Clair feature extraction method is not complete enough, and the accuracy of data analysis processing based on gene features is thereby reduced.

In summary, the above implementation methods of performing feature extraction using Deepvariant and Clair convert a variation detection problem into an identification problem of a two-dimensional image. An artificial intelligence Al model used by Deepvariant is implemented based on a convolutional neural network (CNN), and Clair is implemented based on a long-short term memory artificial neural network (LSTM). However, since both the implementation methods of Deepvariant and Clair transform the sequencing information into information involving three dimensions with each dimension being related to each other, gene information is not fully utilized when only a two-dimensional model is used to extract gene features. Therefore, how to effectively extract and utilize dimensional features is also important.

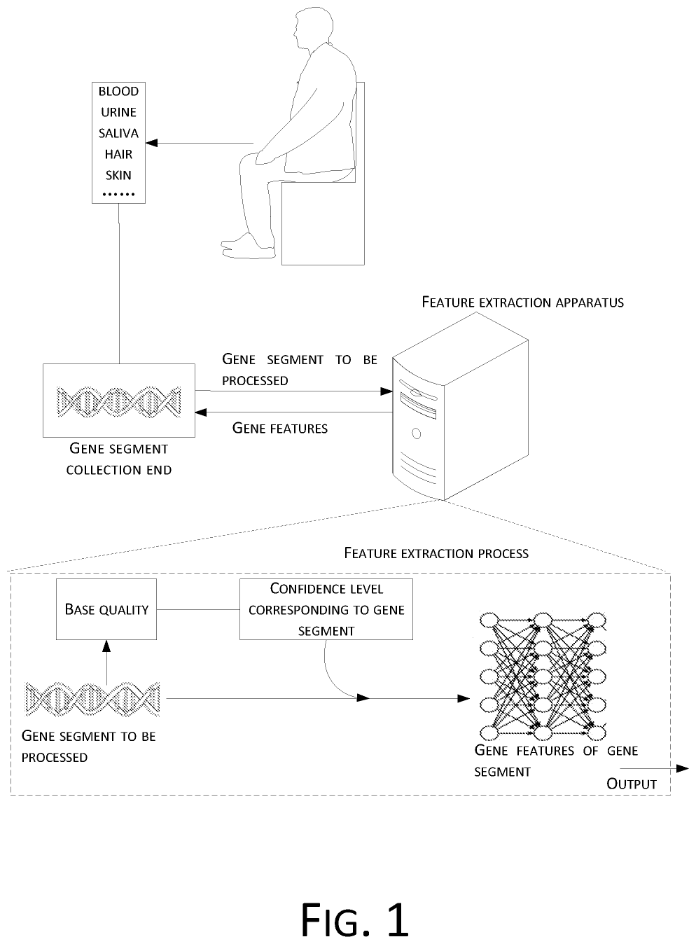

In order to solve the above technical problems, this embodiment proposes methods, apparatuses, and devices for genome feature extraction and disease prediction. An execution subject of the method for genome feature extraction may be a genome feature extraction apparatus. A gene segment collection end may be disposed on the gene feature extraction apparatus. Alternatively, the genome feature extraction apparatus may be communicatively connected to the gene segment collection end, as shown in FIG. 1:

The gene segment collection end can be any computing device having certain gene segment transmission capability and gene segment collection capability. In specific implementations, the gene segment collection end may be a blood collector, a saliva collector, a skin collector and the like. In addition, the basic structure of the gene segment collection end may include: at least one processor. The number of processors depends on the configuration and the type of the gene segment collection end. The gene segment collection end may also include a memory, which may be volatile, such as RAM, or non-volatile, such as read-only memory (ROM), flash memory, etc., or may include both types. The memory typically stores an operating system (abbreviated as OS) and one or more application programs, and may also store program data, etc. Other than processing unit(s) and memory, the gene segment collection end also includes some basic configurations, such as a network card chip, an IO bus, a display component, and some peripheral devices, etc. Optionally, the peripheral devices may include, for example, a keyboard, a mouse, a stylus, a printer, and the like. Other peripheral devices are well known in the art, and will not be described in detail herein.

The genome feature extraction apparatus is a device that can provide a genome feature extraction service in a network virtual environment, and is generally an apparatus that performs information planning and genome feature extraction operations using a network. In physical implementations, the genome feature extraction apparatus may be any device capable of providing computing services, responding to service requests, and performing processing. Examples may be a cluster server, a regular server, a cloud server, a virtual center, etc. The genome feature extraction apparatus mainly includes a processor, a hard disk, a memory, a system bus, etc., and is similar to a general computer framework.

In the above embodiment, the gene segment collection end may conduct a network connection with the genome feature extraction apparatus via a network. Such network connection may be a wireless or wired network connection. If the gene segment collection end is connected with the genome feature extraction apparatus via communication, a network format of such mobile network may be any one of 2G (GSM), 2.5G (GPRS), 3G (WCDMA, TD-SCDMA, CDMA2000, UTMS), 4G (LTE), 4G +(LTE+), WiMax, 5G, and the like.

In this embodiment of the present disclosure, the gene segment collection end may collect a gene segment to be processed by obtaining a set object (a person, an animal, etc.). The gene segment includes a base quality. After the gene segment to be processed is obtained, the gene segment to be processed may be uploaded to the genome feature extraction apparatus, so that the feature extraction apparatus may analyze the uploaded gene segment to be processed.

The genome feature extraction apparatus is used for receiving a gene segment to be processed uploaded by the gene segment collection end. The feature extraction apparatus may then perform a feature extraction operation on the gene segment, so that gene features of the gene segment can be obtained. Specifically, after the gene segment to be processed is obtained, a confidence level corresponding to the gene segment may be determined based on the quality of the bases included in the gene segment. A feature extraction operation may then be performed on the gene segment based on the confidence level corresponding to the gene segment, so that gene features of the gene segment may be accurately and effectively obtained.

According to the technical solutions provided by this embodiment, a gene segment to be processed is obtained, a confidence level corresponding to the gene segment is determined based on the quality of the bases included in the gene segment. A feature extraction operation is performed on the gene segment based on the confidence level corresponding to the gene segment to obtain gene features of the gene segment, thus effectively integrating the quality of the bases into the gene features without increasing the dimensionality of data. As such, not only the mode of implementation is simple and reliable, and the completeness of extraction of the gene features is ensured, but also the operation efficiency of the extraction operation on the gene features is improved, thus further improving the practicability of the technical solutions.

Some implementations of the present disclosure are described in detail below with reference to the accompanying drawings. On condition that there is no conflict between various embodiments, the following embodiments and the characteristics of the following embodiments may be combined with each other.

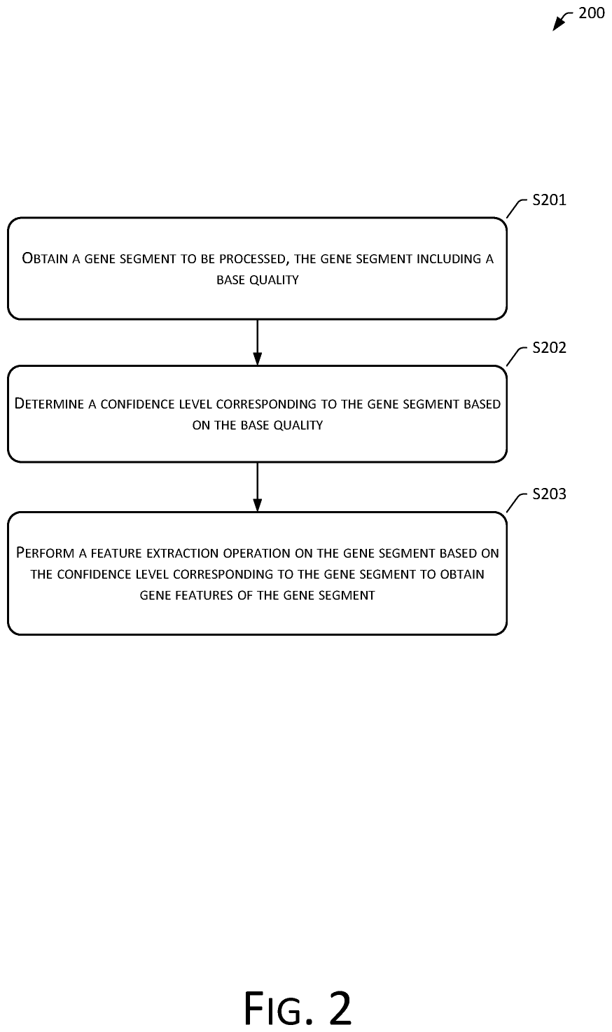

FIG. 2 is a schematic flowchart of a genome feature extraction method 200 according to the embodiments of the present disclosure. Referring to FIG. 2, this embodiment provides a genome feature extraction method 200, where an execution subject of the method may be a genome feature extraction apparatus. It is understood that the genome feature extraction apparatus may be implemented as software, or a combination of software and hardware. Specifically, the feature extraction method 200 may include the following steps:

Step S201: Obtain a gene segment to be processed, the gene segment including a base quality.

Step S202: Determine a confidence level corresponding to the gene segment based on the base quality.

Step S203: Perform a feature extraction operation on the gene segment based on the confidence level corresponding to the gene segment to obtain gene features of the gene segment.

The above steps are explained in detail below:

Step S201: Obtain a gene segment to be processed, the gene segment including a base quality.

A gene segment to be processed refers to a gene segment which needs to be subjected to a feature extraction operation, and the gene segment may include a base quality. It is understood that the gene segment may include not only the base quality as described above, but also other information. For example, the gene segment may include information such as base information (A, C, G, T), a mapping quality, strands (A, C, G, T, A−, C−, G−, T−, wherein the latter four strands are negative strands and the former four strands are positive strands), etc.

In addition, this embodiment does not limit a specific method of obtaining the gene segment. For example, the gene segment to be processed may be stored in a set region, and the gene segment may be obtained by accessing the set region. In other examples, the feature extraction apparatus is provided with a gene collection module, and gene segment(s) can be obtained through the gene collection module. In different application scenes, the gene collection module may correspond to different structural features. For example, when obtaining the gene segment to be processed through blood, the gene collection module may be a blood collector. Specifically, a blood detector collects blood from the body of a set object (person, animal, etc.), and extracts the gene segment to be processed based on the blood. Similarly, when the gene segment to be processed is obtained through saliva, the gene collection module may be a saliva collector. Specifically, the blood detector collects saliva from the body of a set object (person, animal, etc.), and extracts the gene segment to be processed from the saliva. Similarly, when the gene segment to be processed is obtained through skin, the gene collection module may be a skin collector. Specifically, the skin collector collects skin from the body of a set object (person, animal, etc.), and extracts the gene segment to be processed from the skin.

Apparently, one skilled in the art may also use other methods to obtain the gene segment to be processed, as long as the accuracy and reliability of obtaining the gene segment to be processed can be ensured, and details are not described herein.

Step S202: Determine a confidence level corresponding to the gene segment based on the base quality.

Since there is a mapping relationship between the base quality and the confidence level corresponding to the gene segment, after the gene segment to be processed is obtained, the confidence level corresponding to the gene segment can be determined based on the base quality included in the gene segment. In some examples, determining the confidence level corresponding to the gene segment based on the base quality may include: obtaining ratio information between the base quality and 10; and determining the confidence level corresponding to the gene segment based on the ratio information, wherein the confidence level is positively correlated with the base quality and is less than one.

Specifically, when the base quality (qual) is obtained, the ratio information

$$( \frac{q u a l}{10} )$$

between the base quality (qual) and 10 can be obtained. Then, based on the ratio information

$$( \frac{q u a l}{10} ) ,$$

the confidence level (p) corresponding to the gene segment is determined. In some instances, with the confidence level as

$$p = 1 - 1 {0}^{- \frac{q u a l}{10}} ,$$

the confidence level (p) is a value between 0 and 1, and the confidence level (p) is positively correlated with the base quality, i.e., the greater the base quality, the higher the base quality included in the gene segment is. This indicates that the accuracy of the gene segment is higher, and thus it can determine that the confidence level (p) of the gene segment is also increased. Similarly, the confidence level (p) becomes lower as the base quality becomes lower.

Apparently, one skilled in the art can also employ other ways to obtain the confidence level (p) corresponding to the gene segment. For example, when the confidence level as

$$p = {10}^{- \frac{q u a l}{10}} ,$$

the confidence level is negatively correlated with the base quality. In other words, the confidence level (p) is lower when the base quality is higher, and the confidence level (p) becomes higher as the base quality becomes lower.

Step S203: Perform a feature extraction operation on the gene segment based on the confidence level corresponding to the gene segment to obtain gene features of the gene segment.

After the confidence level corresponding to the gene segment is obtained, a feature extraction operation can be performed on the gene segment based on the confidence level corresponding to the gene segment, so that gene features of the gene segment can be obtained. In some examples, performing the feature extraction operation on the gene segment based on the confidence level corresponding to the gene segment to obtain the gene featured of the gene segment may include: performing the feature extraction operation on the gene segment in a statistical counting mode based on the confidence level corresponding to the gene segment to obtain the gene features of the gene segment, wherein the gene features include: base information, base positions, and statistics corresponding to the base information.

Specifically, the base information may include at least one of: A, G, C, T, A−, G−, C−, and T−, wherein base information (A, G, C, T) is a positive strand, base information (A−, G−, C−, T−) is a negative strand, and the statistics corresponding to the base information may include at least one of the following: a statistic of bases being identical to reference bases, a statistic of base insertions, a statistic of base deletions, and a statistic of single nucleotide alternative bases. After the confidence level corresponding to the gene segment is obtained, a feature extraction operation can be performed on the gene segment by adopting a method of statistical technology based on the confidence level corresponding to the gene segment, so that the gene features of the gene segment can be stably obtained with the use of the confidence level corresponding to the gene segment, thereby improving the completeness and the efficiency of extracting the gene features.

According to the genome feature extraction method provided by this embodiment, a gene segment to be processed is obtained, a confidence level corresponding to the gene segment is determined based on the quality of the bases included in the gene segment. A feature extraction operation is performed on the gene segment based on the confidence level corresponding to the gene segment to obtain gene features of the gene segment, thus effectively integrating the quality of the bases into the gene features without increasing the dimensionality of data. As such, not only the mode of implementation is simple and reliable, and the completeness of extraction of the gene features is ensured, but also the operation efficiency of the extraction operation on the gene features is improved, thus further improving the practicability of the technical solutions.

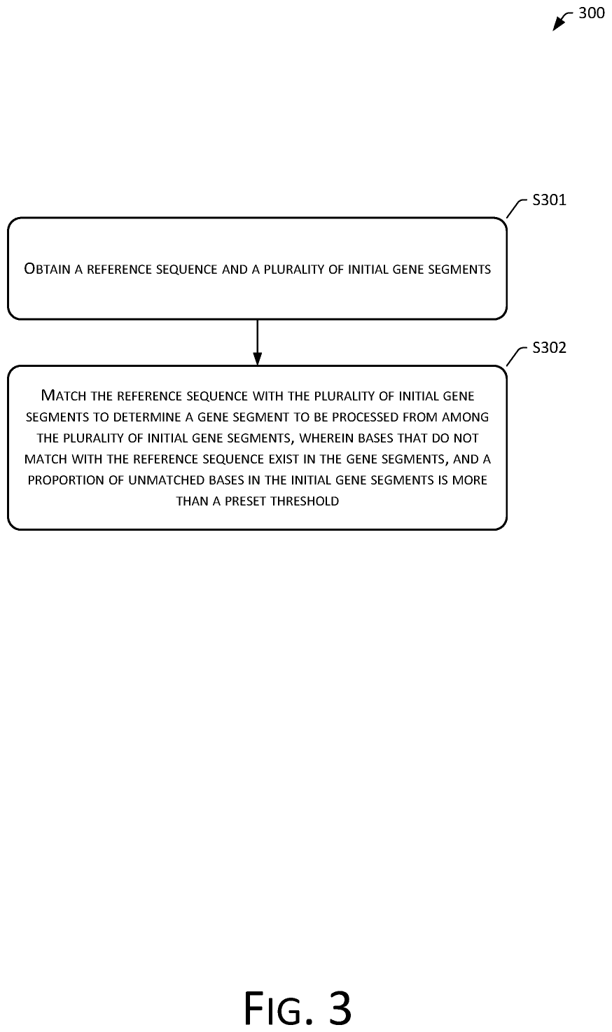

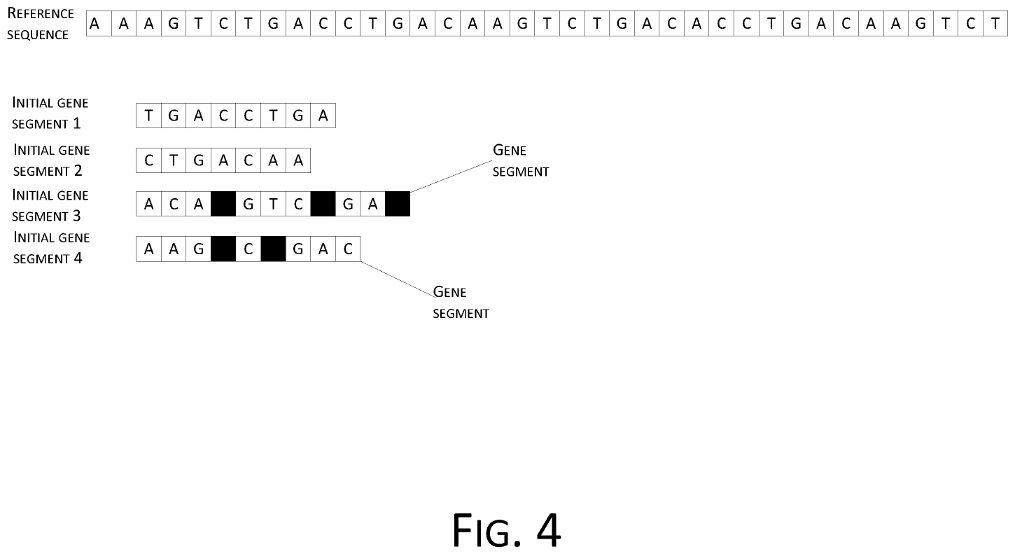

FIG. 3 is a schematic flowchart of obtaining a gene segment to be processed according to the embodiments of the present disclosure; FIG. 4 is a schematic diagram of obtaining a gene segment to be processed according to the embodiments of the present disclosure. On the basis of the above embodiments, referring to FIG. 3 and FIG. 4, this embodiment provides an implementation of obtaining a gene segment to be processed. Specifically, in this embodiment, a method 300 of obtaining a gene segment to be processed may include:

Step S301: Obtain a reference sequence and a plurality of initial gene segments.

Step S302: Match the reference sequence with the plurality of initial gene segments to determine a gene segment to be processed from among the plurality of initial gene segments, wherein bases that do not match with the reference sequence exist in the gene segments, and a proportion of unmatched bases in the initial gene segments is more than a preset threshold.

A reference sequence is standard gene data used for detecting whether an initial gene segment is a gene segment to be processed. A plurality of initial gene segments is gene data which is needed to be detected whether to be a gene segment to be processed. After a plurality of initial gene segments and a reference sequence are obtained, the reference sequence and the plurality of initial gene segments can be analyzed and matched to determine a gene segment to be processed in the plurality of initial gene segments. Specifically, the gene segment to be processed is at least one part of the plurality of initial gene segments. It is noted that bases that do not match with the reference sequence exist in the determined gene segment, and a proportion of unmatched bases in the initial gene segments is greater than a preset threshold.

For example, referring to FIG. 4, the number of initial gene segments is four and the reference sequence is AAAGTCTGACCTGACAAGTCTGACACCTGACAAGTCT are used as an example for illustration. The initial gene segments may include an initial gene segment 1, an initial gene segment 2, an initial gene segment 3 and an initial gene segment 4. The initial gene segment 1 may be TGACCTGA, the initial gene segment 2 may be CTGACAA, the initial gene segment 3 may be ACACGTCAGAT, and the initial gene segment 4 may be AAGGCAGAC.

In order to improve the efficiency of gene feature extraction operations, the initial gene segments may be preliminarily screened, to screen out gene segment(s) with abnormal condition(s) from among the initial gene segments. Specifically, the reference sequence and the initial gene segments can be analyzed and compared. In other words, after obtaining the reference sequence and the initial gene segment 1, the reference sequence and the initial gene segment 1 can be analyzed and matched, the initial gene segment 1 matches with the 12th-19th bases in the reference sequence, i.e., the bases in the initial gene segment 1 completely match with the bases in the reference sequence. This indicates that no gene abnormality exists in the initial gene segment 1, and further explains that the initial gene segment 1 does not satisfy the conditions of a gene segment to be processed. Therefore, the initial gene segment 1 is not determined as a gene segment to be processed.

After the reference sequence and the initial gene segment 2 are obtained, the reference sequence and the initial gene segment 2 can be analyzed and matched. The initial gene segment 2 matches with the 11th-17th bases in the reference sequence. In other words, the bases in the initial gene segment 2 completely match with the bases in the reference sequence. At this time, this indicates that no gene abnormality exists in the initial gene segment 2, and further explains that the initial gene segment 2 does not satisfy the conditions of a gene segment to be processed. Therefore, the initial gene segment 2 is not determined as a gene segment to be processed.

After obtaining the reference sequence and the initial gene segment 3, the reference sequence and the initial gene segment 3 can be analyzed and matched. The initial gene segment 3 matches with the 14th-24th bases in the reference sequence. In other words, the bases in the initial gene segment 3 do not completely match with the bases in the reference sequence. This indicates that a gene abnormality exists in the initial gene segment 3. The number of unmatched bases is 3, and the total number of bases included in the initial gene segment is 11. At this time, the ratio of the unmatched bases in the initial gene segment 3 is 3/11 and is about 0.273. If the preset threshold is 0.1, the ratio of the unmatched bases of the initial gene segment 3 in the initial gene segment is larger than the preset threshold. Specifically, this indicates that the initial gene segment 3 satisfies the conditions of a gene segment to be processed, and the initial gene segment 3 can thus be determined as the gene segment to be processed.

After obtaining the reference sequence and the initial gene segment 4, the reference sequence and the initial gene segment 4 can be analyzed and matched. The initial gene segment 4 matches with the 2nd-10th bases in the reference sequence. In other words, the bases in the initial gene segment 4 do not completely match with the bases in the reference sequence. This indicates that a gene abnormality exists in the initial gene segment 4. The number of unmatched bases is 2, and the total number of bases included in the initial gene segment is 9. At this time, the ratio of the unmatched bases in the initial gene segment 4 is 2/9 and is about 0.222. If the preset threshold is 0.1, the ratio of the unmatched bases of the initial gene segment 4 in the initial gene segment is greater than the preset threshold. Specifically, this indicates that the initial gene segment 4 satisfies the conditions of a gene segment to be processed, and the initial gene segment 4 can thus be determined as the gene segment to be processed.

In this embodiment, the reference sequence and the initial gene segments are obtained and are then matched to determine gene segment(s) to be processed from among the initial gene segments, thus effectively achieving a primary screening of the initial gene segments to obtain the gene segment(s) to be processed. This not only guarantees the accuracy and reliability of determining the gene segment(s) to be processed, but also improves the quality and efficiency of analyzing and processing the gene segments.

To improve the utility of this method, after obtaining the gene features of the gene segment, the method in this embodiment may further include: and performing mutation detection on the gene segment based on the gene features to obtain a mutation detection result.

After the gene features are obtained, mutation detection operation can be performed on the gene segments based on the gene features, so that a mutation detection result can be obtained. In some examples, performing mutation detection on the gene segments based on the gene features to obtain the mutation detection result may include: inputting the gene features into a three-dimensional network model to obtain the mutation detection result, wherein the three-dimensional network model is trained to be used for performing mutation detection on the gene segment based on the gene features.

Specifically, a three-dimensional network model is trained in advance. The three-dimensional network model may be generated by using a three-dimensional convolutional neural network (3D CNN) as a backbone model. The three-dimensional network model is used for performing mutation detection operations on gene segments based on gene features. Specifically, after gene features are obtained, the gene features can be inputted into the three-dimensional network model, so that a mutation detection result can be outputted. As such, the quality and the effect of gene mutation detection are effectively guaranteed.

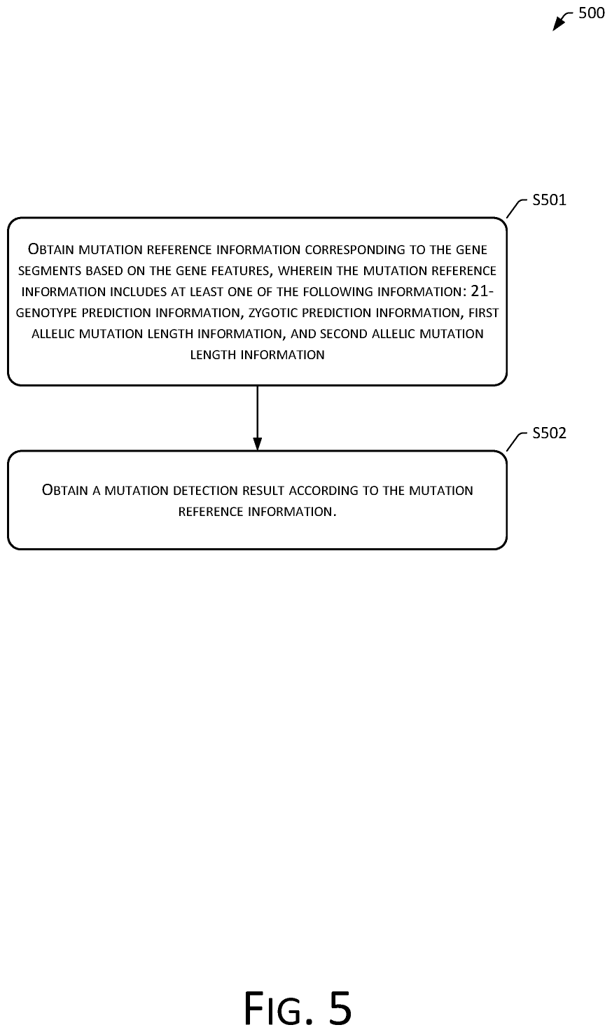

In other examples, referring to FIG. 5, in this embodiment, another implementation of performing mutation detection on a gene segment based on gene features is provided. Specifically, in this embodiment, a method 500 of performing mutation detection on a gene segment based on gene features may include:

Step S501: Obtain mutation reference information corresponding to the gene segments based on the gene features, wherein the mutation reference information includes at least one of the following information: 21-genotype prediction information, zygotic prediction information, first allelic mutation length information, and second allelic mutation length information.

Step S502: Obtain a mutation detection result according to the mutation reference information.

Specifically, after the gene features are obtained, the gene features are analyzed, so that mutation reference information corresponding to the gene segments can be obtained, where the mutation reference information may include at least one of: 21-genotype prediction information, zygote prediction information, first allelic mutation length information, and second allelic mutation length information. The 21 genotypes for which the 21-genotype prediction information is directed include: ‘AA’, ‘AC’, ‘AG’, ‘AT’, ‘CC’, ‘CG’, ‘CT’, ‘GG’, ‘GT’, ‘TT’, ‘AI’, ‘CI’, ‘GI’, ‘TI’, ‘AD’, ‘CD’, ‘GD’, ‘TD’, ‘II’, and ‘DD’, wherein A, C, G, T are four bases, and I and D are insertions and deletions respectively. The zygotic prediction information includes three types: homozygous and identical to reference bases, homozygous and inconsistent with the reference bases, and heterozygous. In the first allelic mutation length information, SNP mutation is 0, and Indel mutation is the length of the corresponding insertions and deletions. In the second allelic mutation length, SNP mutation is 0, and Indel mutation is 0.

After obtaining the mutation reference information corresponding to the gene segment, the mutation reference information may be analyzed to obtain a mutation detection result. It is understood that the mutation detection result is obtained based on at least one of the 21-genotype prediction information, the zygote prediction information, the first allelic mutation length information, and the second allelic mutation length information, thereby ensuring the accuracy and reliability of determining the mutation detection result.

In some examples, after obtaining the mutation detection result, the method of this embodiment may further include: and performing disease prediction based on the mutation detection result.

When the gene segment has a mutation condition, this indicates that the set object is relatively easy to generate related diseases. In this case, disease prediction can be performed based on the mutation detection result. Specifically, probability information of the set object generating a related disease can be determined based on the mutation condition of the gene segment. It can be understood that the probability information is related to the extent of mutation of the gene segment. When the extent of mutation is higher, the probability information is higher. The lower the extent of mutation is, the lower the probability information is. On the other hand, when there is no mutation in the gene segment, this means that a disease is not likely to occur in the set object.

In this embodiment, mutation reference information corresponding to a gene segment is obtained based on gene features, and a mutation detection result is obtained according to the mutation reference information. This not only guarantees the accuracy and reliability of determining a mutation detection result, but also is able to perform disease prediction processing based on an association relationship between the mutation detection result and disease(s) so generated, so that a disease prediction result can be obtained, thus further improving the practicability of this method.

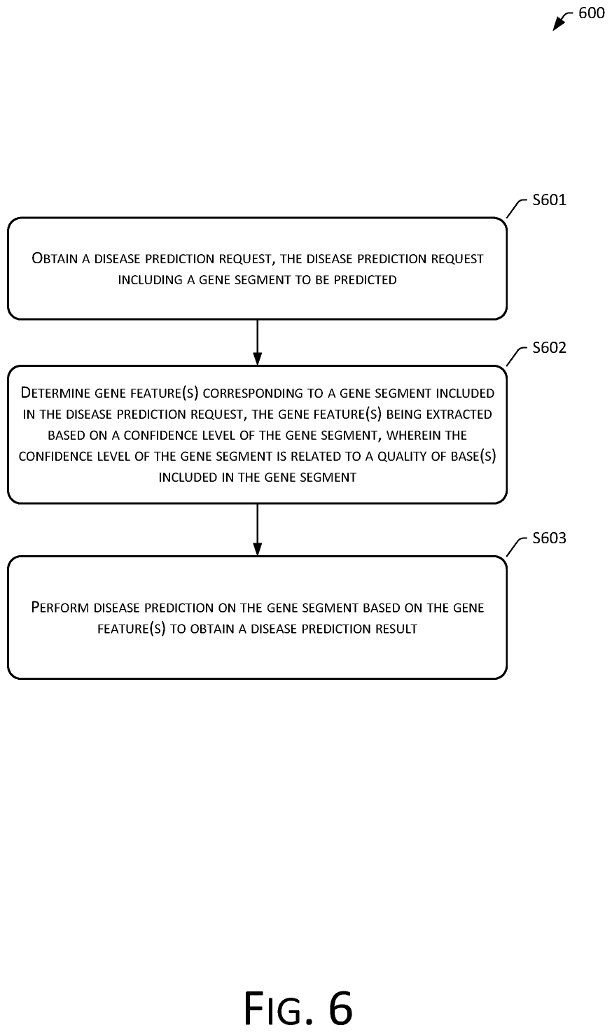

FIG. 6 is a flowchart of a disease prediction method 600 according to the embodiments of the present disclosure. Referring to FIG. 6, this embodiment provides a disease prediction method 600. An execution subject of the method may be a disease prediction apparatus. It can be understood that the disease prediction apparatus may be implemented as software, or a combination of software and hardware. Specifically, the disease prediction method 600 may include the following steps:

Step S601: Obtain a disease prediction request, the disease prediction request including a gene segment to be predicted.

Step S602: Determine gene feature(s) corresponding to a gene segment included in the disease prediction request, the gene feature(s) being extracted based on a confidence level of the gene segment, wherein the confidence level of the gene segment is related to a quality of base(s) included in the gene segment.

Step S603: Perform disease prediction on the gene segment based on the gene feature(s) to obtain a disease prediction result.

The above steps are explained in detail below:

Step S601: Obtain a disease prediction request, the disease prediction request including a gene segment to be predicted.

The disease prediction request includes a gene segment to be predicted, and the disease prediction request is used for realizing a disease prediction operation based on the gene segment to be predicted. Specifically, this embodiment does not limit specific implementations of obtaining a disease prediction request. For example, a disease prediction request may be stored in a preset region, and the disease prediction request may be obtained by accessing the preset region. Alternatively, the disease prediction apparatus may be provided with an interactive interface in which a user may input an execution operation, so that the disease prediction apparatus may generate a disease prediction request based on the execution operation. Alternatively, the disease prediction apparatus may be communicatively connected to a client, and the client may transmit a disease prediction request to the disease prediction apparatus after generating the disease prediction request, so that the disease prediction apparatus can stably obtain the disease prediction request.

Apparently, specific implementations of obtaining a disease prediction request are not limited to the above description, and one skilled in the art may also obtain the disease prediction request in other manners as long as the accuracy and reliability of obtaining the disease prediction request can be ensured, which is not described herein again.

Step S602: Determine gene feature(s) corresponding to a gene segment included in the disease prediction request, the gene feature(s) being extracted based on a confidence level of the gene segment, wherein the confidence level of the gene segment is related to a quality of base(s) included in the gene segment.

After the disease prediction request is obtained, the gene segment included in the disease prediction request may be analyzed to determine gene feature(s) corresponding to the gene segment. The gene feature(s) is/are extracted based on a confidence level of the gene segment, and the confidence level of the gene segment is related to the quality of bases included in the gene segment. Specifically, specific implementation processes and implementation effects of determining gene feature(s) corresponding to a gene segment included in a disease prediction request in this embodiment are similar to specific implementation processes and implementation effects of steps S201 to S203 in the foregoing embodiments. Details may be referenced to the above description, which are not repeated herein.

Step S603: Perform disease prediction on the gene segment based on the gene feature(s) to obtain a disease prediction result.

After the gene feature(s) are obtained, mutation detection can be performed on the gene segment based on the gene feature(s) to obtain a mutation detection result, and disease prediction can then be performed based on the mutation detection result, so that a disease prediction result can be obtained. Specifically, specific implementation processes and implementation effects of “performing disease prediction on the gene segment based on the gene feature(s) to obtain a disease prediction result” in this embodiment are similar to the specific implementation processes and implementation effects of “performing mutation detection on the gene segment based on gene feature(s) to obtain a mutation detection result” and “performing disease prediction based on the mutation detection result” in the above embodiments, and details may be referenced to the above description, and are not repeated herein.

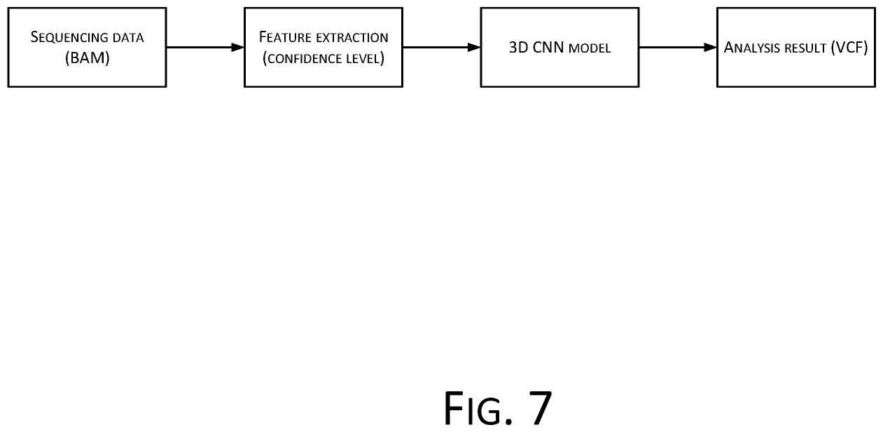

In specific applications, referring to FIG. 7, the embodiments of the present disclosure provides a method for analyzing and processing a genome, in which Clair and Deepvariant methods are organically combined. Specifically, the method includes the following steps:

Step 1: Obtain sequencing data.

The sequencing data may be gene data to be analyzed. The gene data may be a file in a BAM format, and include a large number of sequencing reads segments (corresponding to gene segments in the above embodiments). Each read segment includes information such as base information, a base quality, positive and negative strands, and the like.

Step 2: Determine a confidence level of the sequencing data based on a base quality included in the sequencing data.

Specifically, after the base quality included in the sequencing data is obtained, the base quality corresponding to each base may be converted into a confidence level in a range of 0 to 1, so as to perform a gene feature extraction operation.

Step 3: Perform a feature extraction operation on the sequencing data based on the confidence level of the sequencing data to obtain gene features.

Specifically, after the confidence level of the sequencing data is obtained, a feature extraction operation in a statistical counting manner may be performed on the sequencing data based on the confidence level corresponding to the sequencing data, to obtain gene features of the sequencing data. The gene features include: base information, base positions and statistics corresponding to the base information. In this way, a result of low-quality gene features caused by the problem of sequencing can be effectively avoided.

An example of gene features taken as a three-dimensional feature vector with a size of 33×8×4 is used as follows. The three dimensions of the gene features are respectively used for identification: base positions, all possible base information, and statistics corresponding to the base information, wherein the base positions are 16 positions that extend to the left and right sides with a candidate position as the center. All the possible base information may include: A. C, G, T, A−, C−, G−, and T−, wherein the last four bases are negative strands, the first four bases are positive strands. The statistics corresponding to the base information may include four different statistical methods, which specifically include: a statistic of bases being identical to reference bases, a statistic of insertion cases, a statistic of deletion cases, and a statistic of single nucleotide alternative bases. It is understood that after the sequencing data is obtained, base qualities can be converted into confidence levels, and the confidence levels are then accumulated to obtain a corresponding statistic.

Step 4: input the gene features into a pre-trained 3D CNN network model to obtain a gene analysis result.

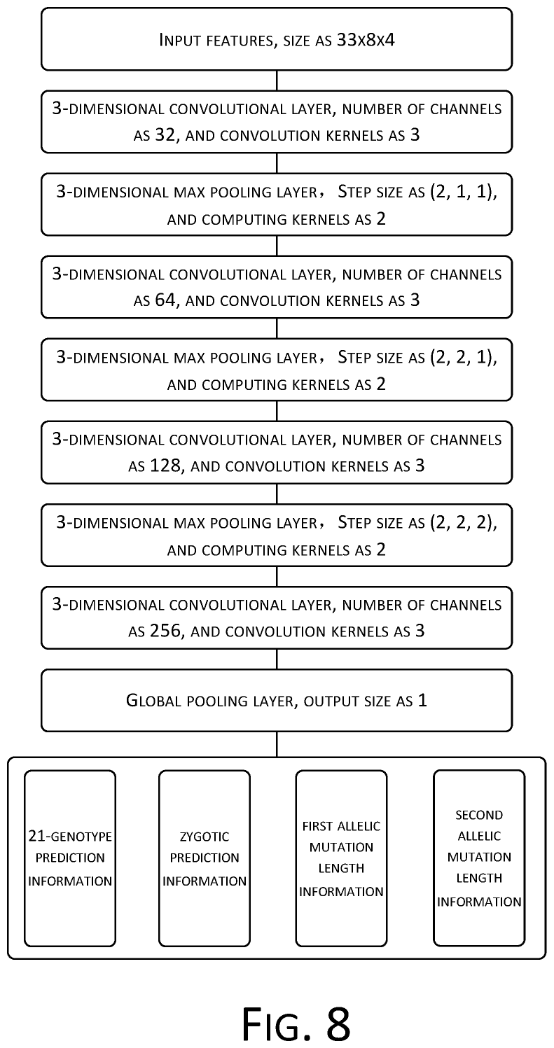

After the gene features are obtained, the gene features can be inputted into a 3D CNN network model. The 3D CNN network model can then analyze and process the gene features to obtain a gene analysis result. For example, when the gene features are a three-dimensional feature vector with a size of 33×8×4, the following describes a process for implementing the analysis processing on the gene features by the 3D CNN network model. Referring to FIG. 8, the 3D CNN network model may include a convolutional layer and a pooling layer, so that the inputted gene features may be analyzed using the convolutional layer and the pooling layer.

In specific implementations, the 3D network model includes four task processing modules, which are respectively: a first processing module used for implementing a 21-genotype probability prediction, a second processing module used for implementing a zygosity probability prediction, a third processing module used for determining the length of a first allelic mutation, and a fourth processing module used for determining the length of a second allelic mutation. After the four task processing modules analyze and process the gene features, four task processing results can be obtained.

Task 1: The first processing module is capable of performing a probability prediction operation for 21 genotypes with respect to the gene features, and the 21 genotypes respectively include: ‘AA’, ‘AC’, ‘AG’, ‘AT’, ‘CC’, ‘CG’, ‘CT’, ‘GG’, ‘GT’, ‘TT’, ‘AI’, ‘CI’, ‘GI’, ‘TI’, ‘AD’, ‘CD’, ‘GD’, ‘TD’, ‘II’, and ‘DD’, wherein A, C, G, T are four bases and I and D are an insertion and a deletion respectively. After the gene features are analyzed and processed by the first processing module, prediction information of 21 genotypes can be obtained.

Task 2: The second processing module is capable of performing a zygotic probability prediction operation on the gene features, where the zygotic probability prediction includes three types of cases, which are: homozygous and identical to reference bases, homozygous and not identical to the reference bases, and heterozygous. After the gene features are analyzed and processed by the second processing module, zygotic prediction information can be obtained.

Task 3: The third processing module is capable of determining the length of a first allelic mutation according to the gene features. When SNP mutation is 0, Indel mutation is the length of a corresponding insertion/deletion. After the analysis processing is performed on the gene features using the third processing module, a first piece of allelic mutation length information can be obtained.

Task 4: The fourth processing module is capable of determining the length of a second allelic mutation for the gene feature. When SNP mutation is 0, Indel mutation is the length of a corresponding insertion/deletion. After the fourth processing module is used to analyze and process the gene features, a second piece of allelic mutation length information can be obtained.

After the four task processing results are obtained, a gene analysis result may be determined based on the four task processing results. In short, the four task processing results can be comprehensively analyzed, so that a gene analysis result can be obtained. For example, if a result of gene analysis of task 1 indicates that a mutation result of the gene segment is a SNP type, the lengths predicted in tasks 3 and 4 are 0. Specifically, a specific strategy for determining a gene analysis result based on the four task processing results is not limited in this embodiment of the present disclosure. One skilled in the art can adjust and configure the strategy according to specific application requirements and design requirements, which are not repeatedly described herein.

According to the genome analysis processing method provided by the embodiments of the present disclosure, a confidence level of a gene segment is determined based on the base qualities of the gene segment, and a feature extraction operation is then performed based on the confidence level and using Clair statistics to obtain gene features. As such, the base qualities are effectively integrated into the gene features without increasing the dimensionality of data. This not only guarantees the completeness of the gene features, but also ensures the efficiency of the feature extraction operation. In addition, after the gene features are obtained, the gene features are inputted into a 3D CNN, and the gene features that are extracted are analyzed and processed using the 3D CNN network model, so that the three-dimensional features are fully used for obtaining gene analysis results, and the accuracy of analysis and processing of gene data is further enhanced.

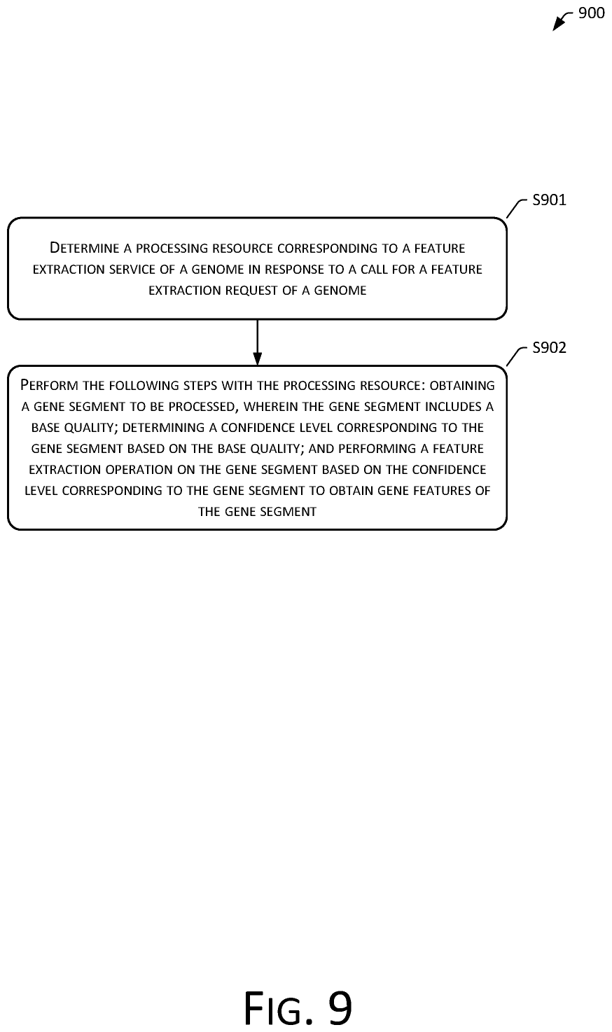

FIG. 9 is a schematic flowchart of a genome feature extraction method according to the embodiments of the present disclosure. Referring to FIG. 9, this embodiment provides a genome feature extraction method 900, where the execution subject of the genome feature extraction method may be a genome feature extraction apparatus. It is understood that the genome feature extraction apparatus may be implemented as software or a combination of software and hardware. Specifically, the genome feature extraction method 900 may include:

Step S901: Determine a processing resource corresponding to a feature extraction service of a genome in response to a call for a feature extraction request of a genome.

Step S902: Perform the following steps with the processing resource: obtaining a gene segment to be processed, wherein the gene segment includes a base quality; determining a confidence level corresponding to the gene segment based on the base quality; and performing a feature extraction operation on the gene segment based on the confidence level corresponding to the gene segment to obtain gene features of the gene segment.

Specifically, the genome feature extraction method provided by the present disclosure can be executed in a cloud. A plurality of computing nodes can be deployed in the cloud, and each computing node has processing resources such as computing, storage and the like. In the cloud, multiple computing nodes may be organized to provide a certain service. Apparently, a single computing node may also provide one or more services.

According to the solutions provided by the present disclosure, the cloud end can be provided with a service for completing a method of extracting features of a genome, namely a feature extraction service of genomes. When a user needs to use the feature extraction service of genomes, the feature extraction service of genomes is called so as to trigger a request for calling the feature extraction service of genomes to the cloud. The request may include genome information to be processed. The cloud determines a computing node that responds to the request, and performs the following steps using processing resources in the computing node: obtaining a gene segment to be processed, the gene segment including a base quality; determining a confidence level corresponding to the gene segment based on the base quality; and performing a feature extraction operation on the gene segment based on the confidence level corresponding to the gene segment to obtain gene features of the gene segment.

Specifically, implementation processes, implementation principles and implementation effects of the above method steps in this embodiment are similar to the implementation processes, implementation principles and implementation effects of the method steps in the embodiments shown in FIG. 1-FIG. 5, FIG. 7, and FIG. 8. Reference may be made to related description of the embodiments shown in FIG. 1-FIG. 5, FIG. 7, and FIG. 8 for parts that are not described in detail in this embodiment.

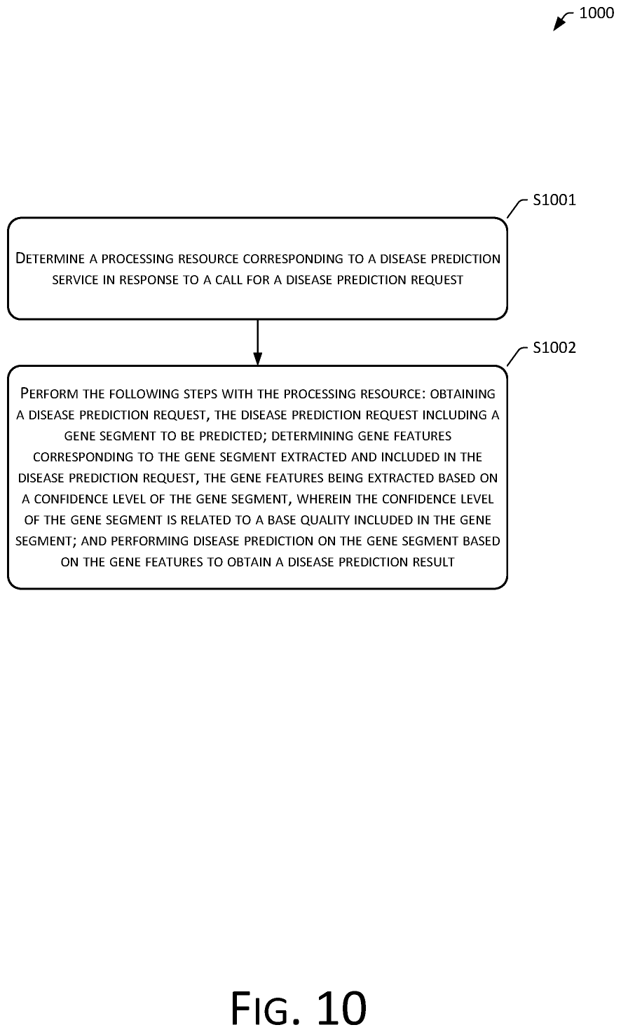

FIG. 10 is a flowchart illustrating a disease prediction method according to the embodiments of the present disclosure. Referring to FIG. 10, this embodiment provides a disease prediction method 1000, and an execution subject of the disease prediction method may be a disease prediction apparatus. It is understood that the disease prediction apparatus may be implemented as software, or a combination of software and hardware. Specifically, the disease prediction method 1000 may include:

Step S1001: Determine a processing resource corresponding to a disease prediction service in response to a call for a disease prediction request.

Step S1002: Perform the following steps with the processing resource: obtaining a disease prediction request, the disease prediction request including a gene segment to be predicted; determining gene features corresponding to the gene segment extracted and included in the disease prediction request, the gene features being extracted based on a confidence level of the gene segment, wherein the confidence level of the gene segment is related to a base quality included in the gene segment; and performing disease prediction on the gene segment based on the gene features to obtain a disease prediction result.

Specifically, the disease prediction method provided by the present disclosure can be executed in a cloud. A plurality of computing nodes can be deployed in the cloud, and each computing node has processing resources such as computation and storage, etc. In the cloud, multiple computing nodes may be organized to provide a service. Apparently, a single computing node may also provide one or more services.

According to the solutions provided by the present disclosure, the cloud can provide a service for completing a disease prediction method, which is called a disease prediction service. When a user needs to use the disease prediction service, the disease prediction service is called to trigger a request for calling the disease prediction service to the cloud, and the request can include genome information to be processed. The cloud determines a computing node that responds to the request, and performs the following steps using processing resources in the computing node: obtaining a disease prediction request, the disease prediction request including a gene segment to be predicted; determining gene features corresponding to the gene segment extracted and included in the disease prediction request, the gene features being extracted based on a confidence level of the gene segment, wherein the confidence level of the gene segment is related to a base quality included in the gene segment; and performing disease prediction on the gene segment based on the gene features to obtain a disease prediction result.

Specifically, implementation processes, implementation principles and implementation effects of the above method steps in this embodiment are similar to the implementation processes, implementation principles and implementation effects of the method steps in the embodiments shown in FIG. 6-FIG. 8. Reference may be made to related description of the embodiments shown in FIG. 6-FIG. 8 for parts that are not described in detail in this embodiment.

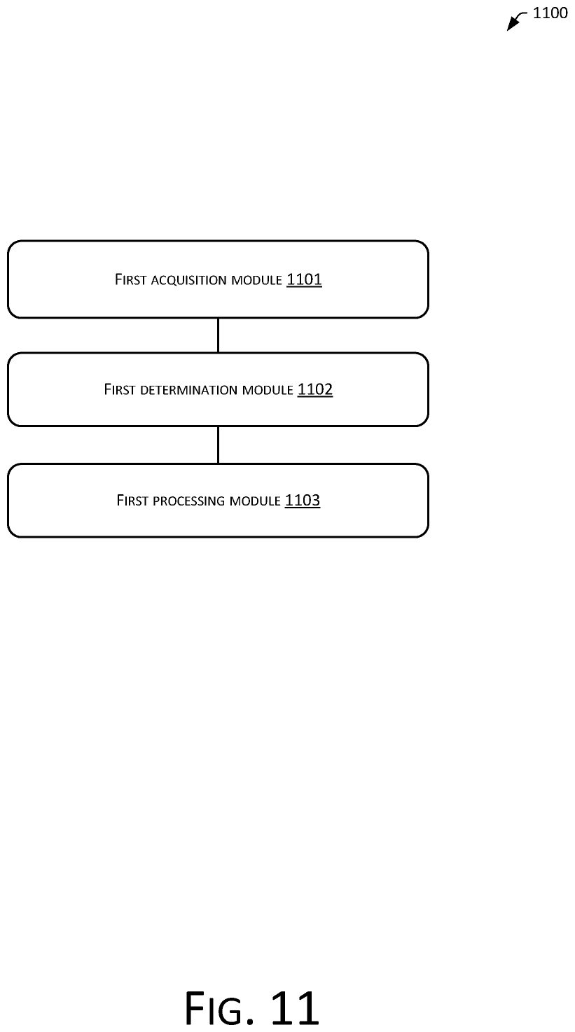

FIG. 11 is a schematic structural diagram of a genome feature extraction apparatus according to the embodiments of the present disclosure. Referring to FIG. 11, this embodiment provides a genome feature extraction apparatus 1100, which can perform the above-mentioned genome feature extraction method as shown in FIG. 2. The genome feature extraction apparatus 1100 may include: a first acquisition module 1101, a first determination module 1102 and a first processing module 1103. Specifically:

the first acquisition module 1101 is configured to obtain a gene segment to be processed, where the gene segment includes a base quality;

the first determination module 1102 is configured to determine a confidence level corresponding to the gene segment based on the base quality; and

the first processing module 1103 is configured to perform a feature extraction operation on the gene segment based on the confidence level corresponding to the gene segment to obtain gene features of the gene segment.

In some examples, when the first acquisition module 1101 obtains the gene segment to be processed, the first acquisition module 1101 is configured to perform: obtaining a reference sequence and a gene sequence, wherein the gene sequence includes a plurality of initial gene segments; and matching the reference sequence with the gene sequence to determine the gene segment to be processed from among the plurality of initial gene segments, wherein bases which do not match with the reference sequence exist in the gene segments, and a ratio of unmatched bases in the gene segments is larger than a preset threshold.

In some examples, when the first determination module 1102 determines the confidence level corresponding to the gene segment based on the base quality, the first determination module 1102 is configured to perform: obtaining ratio information between the base quality and 10; and determining the confidence level corresponding to the gene segment based on the ratio information, wherein the confidence level is positively correlated with the base quality and is less than 1.

In some examples, when the first processing module 1103 performs the feature extraction operation on the gene segment based on the confidence level corresponding to the gene segment to obtain the gene features of the gene segment, the first processing module 1103 is configured to perform: performing the feature extraction operation on the gene segment in a statistical counting mode based on the confidence level corresponding to the gene segment to obtain the gene features of the gene segment, wherein the gene features include base information, base positions, and statistics corresponding to the base information.

In some examples, after obtaining the gene features of the gene segments, the first processing module 1103 in this embodiment is configured to perform: performing mutation detection on the gene segment based on the gene features to obtain a mutation detection result.

In some examples, when the first processing module 1103 performs the mutation detection on the gene segment based on the gene features to obtain the mutation detection result, the first processing module 1103 is configured to perform: inputting the gene features into a three-dimensional network model, and obtaining the mutation detection result, wherein the three-dimensional network model is trained to be used for performing mutation detection on the gene segment based on the gene features.

In some examples, when the first processing module 1103 performs the mutation detection on the gene segment based on the gene features, the first processing module 1103 is configured to perform: obtaining mutation reference information corresponding to the gene segment based on the gene features, wherein the mutation reference information includes at least one of the following information: 21-genotype prediction information, zygotic prediction information, first allelic mutation length information, and second allelic mutation length information; and obtaining the mutation detection result according to the mutation reference information.

In some examples, after obtaining the mutation detection result, the first processing module 1103 in this embodiment is configured to perform: performing disease prediction based on the mutation detection result.

The apparatus shown in FIG. 11 can perform the method of the embodiments shown in FIGS. 1-5, 7, and 8. The parts of this embodiment that are not described in details can be referenced to related descriptions of the embodiments shown in FIGS. 1-5, 7, and 8. The implementation process and technical effect of the technical solution refer to the descriptions in the embodiments shown in FIGS. 1-5, 7, and 8, and are not repeatedly described herein.

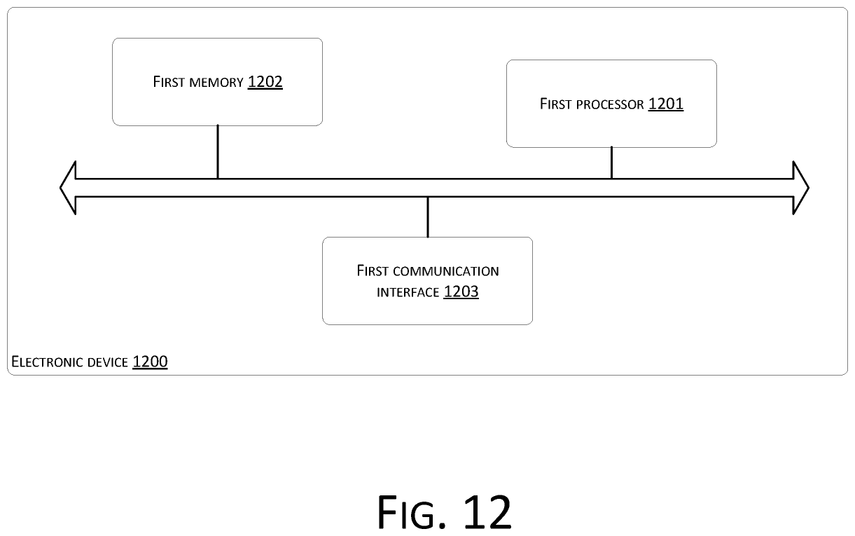

In a possible design, the structure of the genome feature extraction apparatus shown in FIG. 11 may be implemented as an electronic device. The electronic device may be a variety of devices such as a mobile phone, a tablet computer, a server, etc. As shown in FIG. 12, an electronic device 1200 may include: a first processor 1201 and a first memory 1202. The first memory 1202 is used for storing corresponding program(s) of the electronic device for executing the genome feature extraction methods drawn and suggested by the embodiments shown in FIGS. 1-5, 7, and 8. The first processor 1201 is configured for executing the program stored in the first memory 1202.

The program includes one or more computer instructions, wherein the one or more computer instructions, when executed by the first processor 1201, are capable of performing the following steps:

obtaining a gene segment to be processed, the gene segment including a base quality;

determining a confidence level corresponding to the gene segment based on the base quality; and

performing a feature extraction operation on the gene segment based on the confidence level corresponding to the gene segment to obtain gene features of the gene segment.

Furthermore, the first processor 1201 is further configured to execute all or part of the steps in the embodiments shown in FIGS. 1-5, 7, and 8.

The electronic device 1200 may further include a first communication interface 1203 used for communicating with other devices or a communication network.

In addition, the embodiments of the present disclosure provide a computer storage medium for storing computer software instructions for an electronic device, which includes a program for executing the genome feature extraction methods in the method embodiments shown in FIGS. 1-5, 7, and 8.

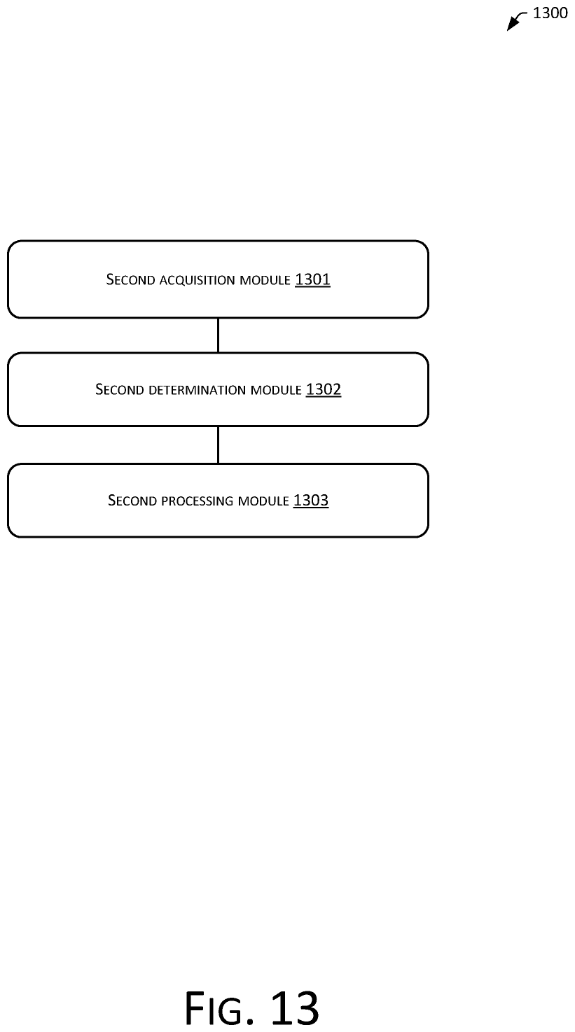

FIG. 13 is a schematic structural diagram of a disease prediction apparatus according to the embodiments of the present disclosure. Referring to FIG. 13, this embodiment provides a disease prediction apparatus 1300 that can perform the disease prediction method shown in FIG. 6, which may include: a second acquisition module 1301, a second determination module 1302 and a second processing module 33. Specifically:

the second acquisition module 1301 is configured to obtain a disease prediction request, the disease prediction request including a gene segment to be predicted;

the second determination module 1302 is configured to determine gene features corresponding to the gene segment included in the disease prediction request, the gene feature being extracted and obtained based on a confidence level of the gene segment, wherein the confidence level of the gene segment is related to qualities of bases included in the gene segment; and

the second processing module 1303 is configured to perform disease prediction on the gene segment based on the gene features to obtain a disease prediction result.

The apparatus shown in FIG. 13 can perform the methods of the embodiments shown in FIGS. 6-8. The parts of this embodiment that are not described in details can be referenced to related descriptions of the embodiments shown in FIGS. 6-8. The implementation process and technical effect of the technical solution refer to the descriptions in the embodiments shown in FIGS. 6-8, and are not repeatedly described herein.

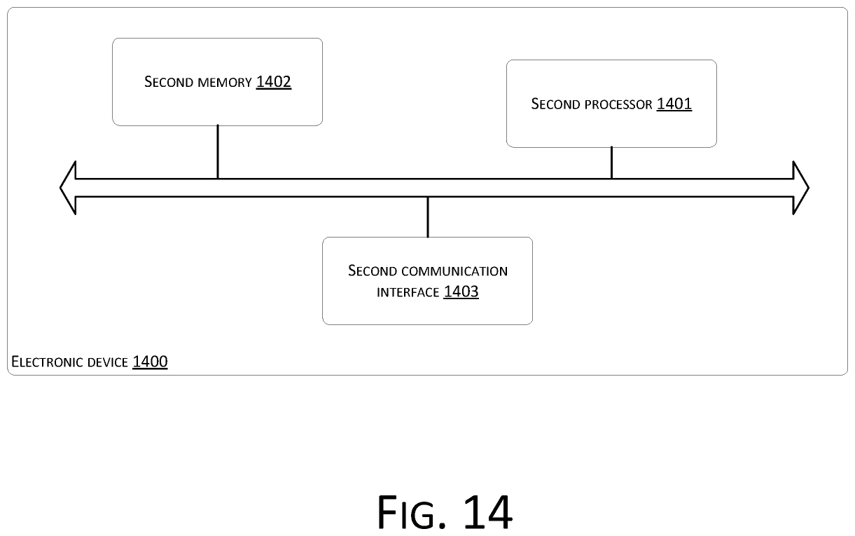

In a possible design, the structure of the disease prediction apparatus shown in FIG. 13 may be implemented as an electronic device. The electronic device may be a variety of devices, such as a mobile phone, a tablet computer, a server, etc. As shown in FIG. 14, an electronic device 1400 may include: a second processor 1401 and a second memory 1402. The second memory 1402 is configured to store a corresponding program of the electronic device for executing the disease prediction method provided in the embodiment shown in FIG. 6, and the second processor 1401 is configured to execute the program stored in the second memory 1402.

The program includes one or more computer instructions, wherein the one or more computer instructions, when executed by the second processor 1401, are capable of performing the following steps:

obtaining a disease prediction request, the disease prediction request including a gene segment to be predicted;

determining gene features corresponding to a gene segment included in the disease prediction request, the gene feature being extracted and obtained based on a confidence level of the gene segment, wherein the confidence level of the gene segment is related to base quality(ies) included in the gene segment; and

performing disease prediction on the gene segment based on the gene features to obtain a disease prediction result.

Furthermore, the second processor 1401 is also configured to execute all or part of the steps in the embodiment shown in FIG. 6.

The electronic device 1400 may further include a second communication interface 1403 used for communicating with other devices or a communication network.

In addition, the embodiments of the present disclosure provide a computer storage medium configured to store computer software instructions for an electronic device, which includes a program for executing the disease prediction method in the method embodiment shown in FIG. 6.

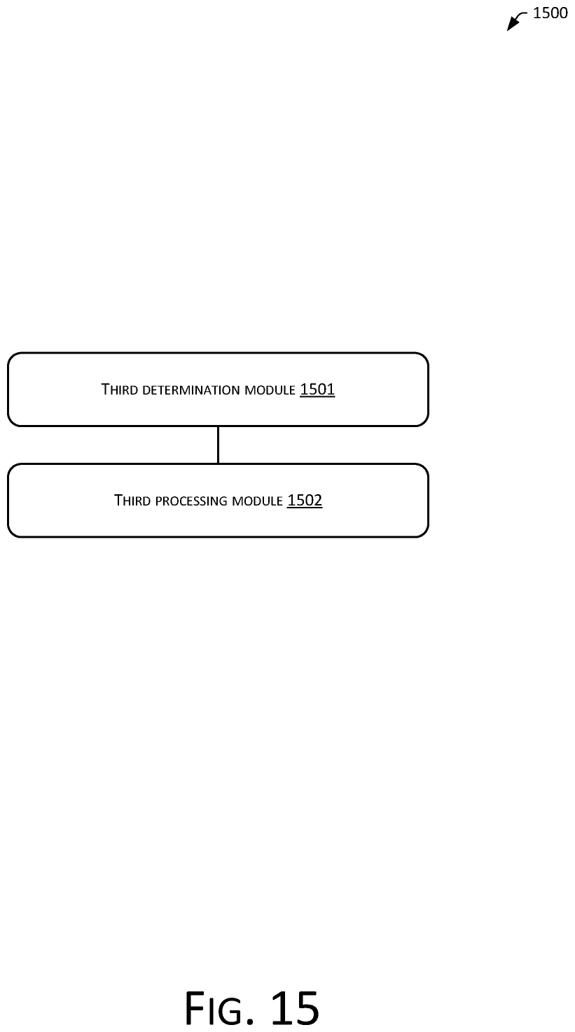

FIG. 15 is a schematic structural diagram of another genome feature extraction apparatus according to the embodiments of the present disclosure. Referring to FIG. 15, this embodiment provides another genome feature extraction apparatus 1500, which can perform the above-mentioned genome feature extraction method shown in FIG. 9, and the genome feature extraction apparatus 1500 may include: a third determination module 1501 and a third processing module 1502. Specifically:

the third determination module 1501 is configured to determine a processing resource corresponding to a genome feature extraction service in response to a call for a feature extraction request of a genome; and

the third processing module 1502 is configured to perform the following steps with the processing resource: obtaining a gene segment to be processed, the gene segment including qualities of bases; determining a confidence level corresponding to the gene segment based on the qualities of the bases; and performing a feature extraction operation on the gene segment based on the confidence level corresponding to the gene segment to obtain gene features of the gene segment.

The apparatus shown in FIG. 15 can execute the method of the embodiments shown in FIG. 9. The parts of this embodiment that are not described in details can be referenced to related descriptions of the embodiments shown in FIG. 9. The implementation process and technical effect of the technical solution refer to the descriptions in the embodiments shown in FIG. 9, and are not repeatedly described herein.

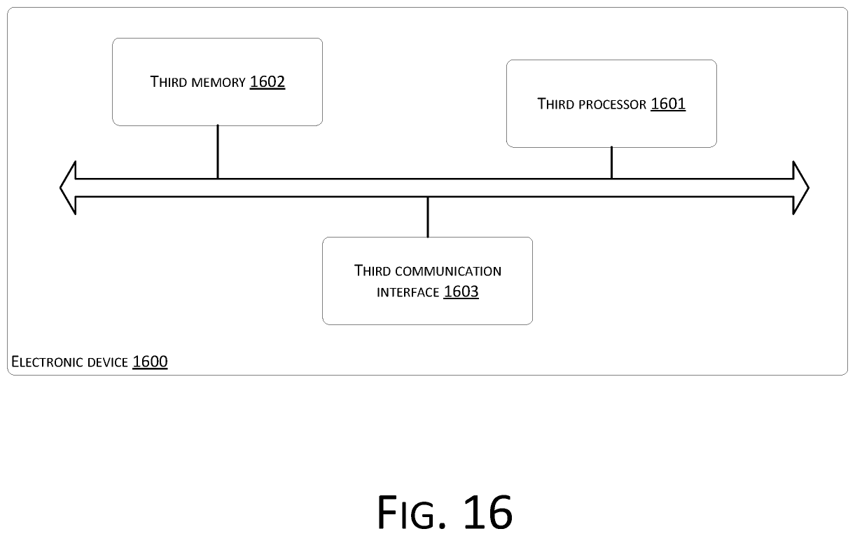

In a possible design, the structure of the genome feature extraction apparatus shown in FIG. 15 may be implemented as an electronic device. The electronic device may be a variety of devices, such as a mobile phone, a tablet computer, a server, etc. As shown in FIG. 16, an electronic device 1600 may include: a third processor 1601 and a third memory 1602. The third memory 1602 is configured to store a corresponding program of the electronic device for executing the genome feature extraction method provided in the embodiments shown in FIG. 9. The third processor 1601 is configured to execute the program stored in the third memory 1602.

The program includes one or more computer instructions, wherein the one or more computer instructions, when executed by the third processor 1601, are capable of performing the following steps:

determine a processing resource corresponding to a genome feature extraction service in response to a call for a feature extraction request of a genome; and

performing the following steps with the processing resource: obtaining a gene segment to be processed, the gene segment including qualities of bases; determining a confidence level corresponding to the gene segment based on the qualities of the bases; and performing a feature extraction operation on the gene segment based on the confidence level corresponding to the gene segment to obtain gene features of the gene segment.

Furthermore, the third processor 1601 is also configured to execute all or part of the steps in the embodiments shown in FIG. 9.

The electronic device 1600 may further include a third communication interface 1603 used for communicating with other devices or a communication network.

In addition, the embodiments of the present disclosure provide a computer storage medium configured to store computer software instructions for an electronic device, which includes a program for executing the genome feature extraction method in the method embodiments shown in FIG. 9.

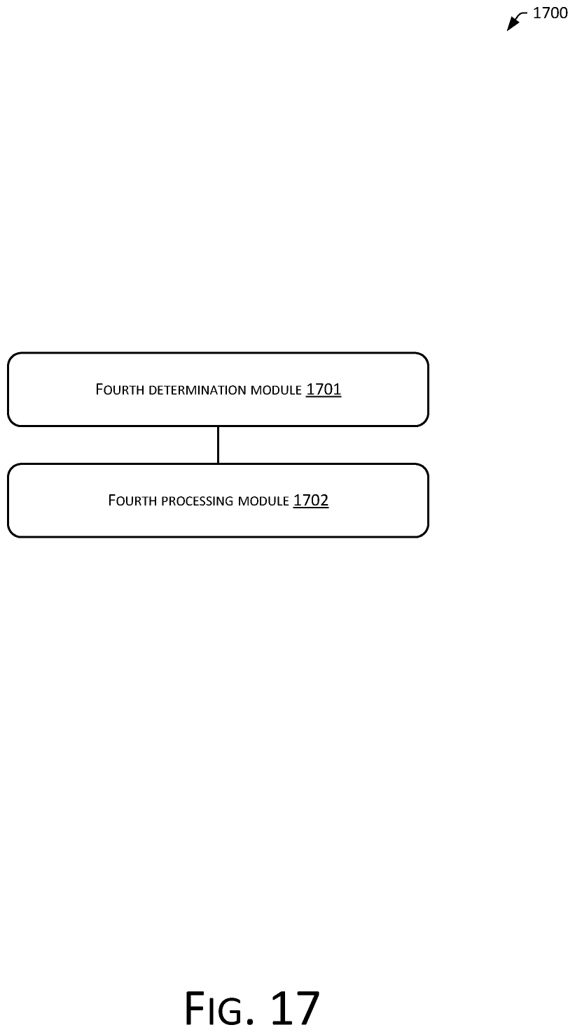

FIG. 17 is a schematic structural diagram of another disease prediction apparatus according to the embodiments of the present disclosure. Referring to FIG. 17, this embodiment provides another disease prediction apparatus 1700 that may perform the disease prediction method shown in FIG. 10. The disease prediction apparatus 1700 may include: a fourth determination module 1701 and a fourth processing module 1702. Specifically:

the fourth determination module 1701 is configured to determine a processing resource corresponding to a disease prediction service in response to a call for a disease prediction request; and

the fourth processing module 1702 is configured to perform the following steps with the processing resource: obtaining a disease prediction request, the disease prediction request including a gene segment to be predicted; determining gene features corresponding to the gene segment included in the disease prediction request, the gene feature being extracted and obtained based on a confidence level of the gene segment, wherein the confidence level of the gene segment is related to qualities of bases included in the gene segment; and performing disease prediction on the gene segment based on the gene features to obtain a disease prediction result.

The apparatus shown in FIG. 17 can execute the method of the embodiments shown in FIG. 9. The parts of this embodiment that are not described in details can be referenced to related descriptions of the embodiments shown in FIG. 10. The implementation process and technical effect of the technical solution refer to the descriptions in the embodiments shown in FIG. 10, and are not repeatedly described herein.

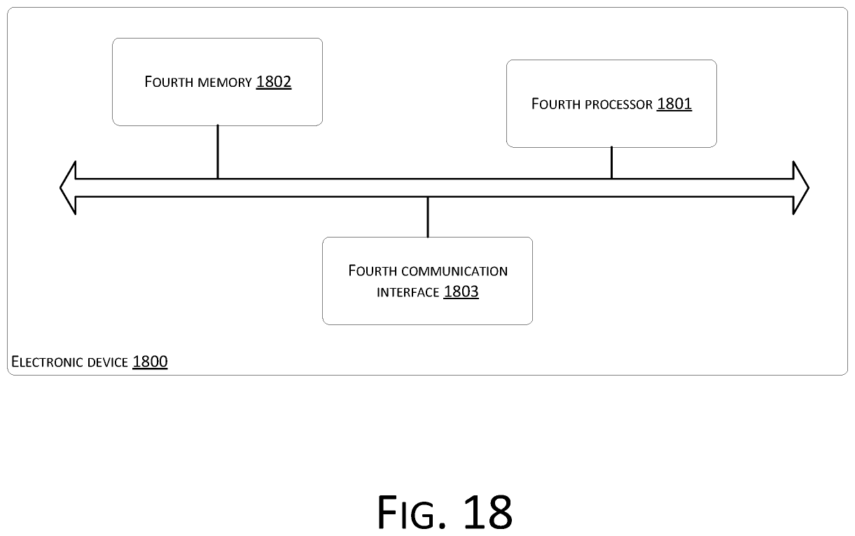

In a possible design, the structure of the disease prediction apparatus shown in FIG. 17 may be implemented as an electronic device. The electronic device may be a variety of devices, such as a mobile phone, a tablet computer, a server, etc. As shown in FIG. 18, an electronic device 1800 may include: a fourth processor 1801 and a fourth memory 1802. The fourth memory 1802 is configured to store a corresponding program of the electronic device for executing the disease prediction method provided in the embodiments shown in FIG. 10. The fourth processor 1801 is configured to execute the program stored in the fourth memory 1802.

The program includes one or more computer instructions, wherein the one or more computer instructions, when executed by the fourth processor 1801, are capable of executing the following steps:

obtaining a disease prediction request, the disease prediction request including a gene segment to be predicted;

determining gene features corresponding to a gene segment included in the disease prediction request, the gene feature being extracted and obtained based on a confidence level of the gene segment, wherein the confidence level of the gene segment is related to qualities of bases included in the gene segment; and

performing disease prediction on the gene segment based on the gene features to obtain a disease prediction result.

Furthermore, the fourth processor 1801 is also configured to execute all or part of the steps in the embodiment shown in FIG. 10.

The electronic device 1800 may further include a fourth communication interface 1803 used for enabling the electronic device to communicate with other devices or a communication network.

In addition, the embodiments of the present disclosure provide a computer storage medium configured to store computer software instructions for an electronic device, which includes a program for executing the disease prediction method in the method embodiments shown in FIG. 10.

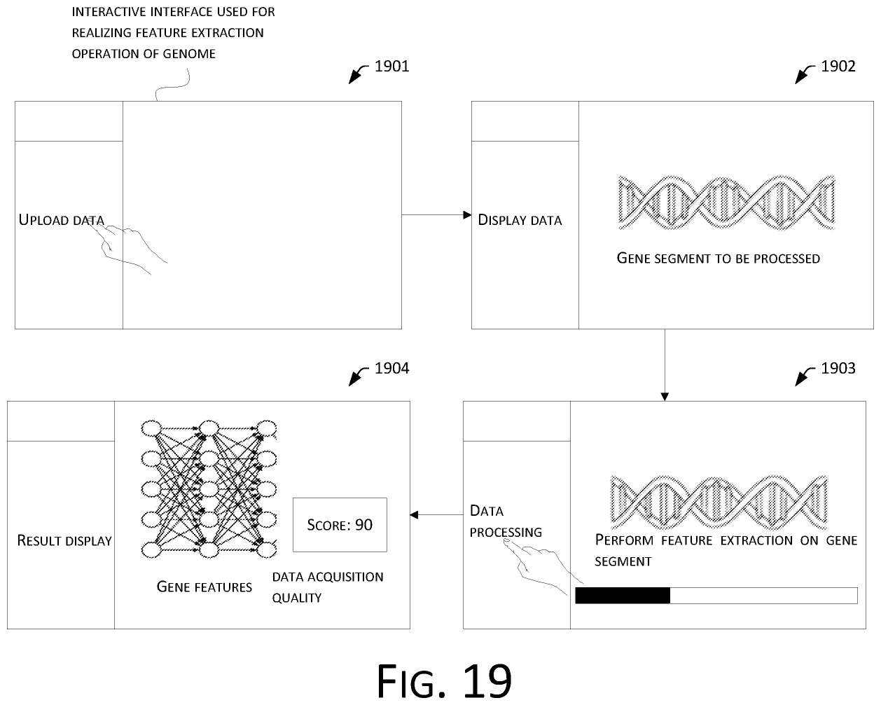

FIG. 19 is a schematic diagram of another genome feature extraction method according to the embodiments of the present disclosure. Referring to FIG. 19, this embodiment provides another genome feature extraction method. An execution subject of the method may be a genome feature extraction apparatus. It is understood that the genome feature extraction apparatus may be implemented as software, or a combination of software and hardware. Specifically, the feature extraction method may include the following steps:

Step S1901: Display an interactive interface for realizing a feature extraction operation of a genome.

When a user has a need for feature extraction of a genome, a service capable of realizing a feature extraction operation on the genome may be called. An interactive interface for realizing the feature extraction operation on the genome is then displayed through a display device. The interactive interface may include a display control for the user to input and execute operations. Specifically, the display control may include: an “upload data” control, and the like.

Step S1902: Obtain a gene segment to be processed in response to an execution operation inputted on the interactive interface, the gene segment including a base quality.

Specifically, after displaying an interactive interface for implementing the feature extraction operation on the genome, the user may input a corresponding execution operation in the interactive interface, for example, a click operation, a slide operation, etc., to implement a corresponding function. For example, when the “upload data” control is displayed on the interactive interface, the user may perform a click operation on the “upload data” control, so that gene segment(s) to be processed (the number may be one or more) may be uploaded to the genome feature extraction apparatus, so that the feature extraction apparatus may perform a feature extraction operation on the gene segment(s) to be processed, where the gene segment(s) may include qualities of bases.

Step S1903: Determine gene features of the gene segment and a data acquisition quality corresponding to the gene features, wherein the gene features are obtained by extraction based on a confidence level of the gene segment, and the confidence level of the gene segment are related to the base quality.

After obtaining the gene segment to be processed, the feature extraction apparatus may perform a feature extraction operation on the gene segment to be processed. Specifically, a “data processing” control can be displayed on the interactive interface, and the user performs a click operation on the “data processing” control, so that the gene features of the gene segment and the data acquisition quality corresponding to the gene features are determined. Specific implementation processes and implementation effects of the determined gene features of the gene segment are similar to those of the embodiments shown in FIG. 2. Details thereof may be referenced to the above description, and are not repeated herein.

The data acquisition quality may be obtained by processing and analyzing the gene features. In some examples, the data acquisition quality may be determined by performing parameter statistics on the gene features. For example, feature statistics may be performed on the gene features to obtain an accumulated value, a mean value and a median value of parameters, and the data acquisition quality is determined based on at least one obtained statistical value.

In addition, when the feature extraction apparatus determines the gene features of the gene segment and the data acquisition quality corresponding to the gene features, in order to enable a user to quickly and timely know the process and progress of the feature extraction, relevant information for identifying the data processing progress can be displayed in the interactive interface. For example, a progress bar associated with the feature extraction operation on the gene segment may be displayed in the interactive interface.

Step S1904: Display the gene features and the data acquisition quality in the interactive interface.

When the feature extraction operation is completed, the gene features and the data acquisition quality corresponding to the gene segment can be obtained. In order to enable the user to know a data processing result, the gene features and the data acquisition quality can be displayed in the interactive interface, wherein the data acquisition quality may be numerical information. For example, when the gene features of the gene segment are obtained, the data acquisition quality corresponding to the gene features is a score of 90.

According to the genome feature extraction method, by displaying an interactive interface for realizing a feature extraction operation on a genome, a gene segment to be processed is obtained in response to an execution operation inputted on the interactive interface. Gene features of the gene segment and a data acquisition quality corresponding to the gene features are determined, and the gene features and the data acquisition quality can be displayed in the interactive interface. In this way, the feature extraction operation is effectively realized by interacting with the user, which not only ensures the quality and the efficiency of the feature extraction operation, but also enables the user to timely know and obtain relevant information of the feature extraction operation, thus effectively improving the degree of convenience of the feature extraction method.

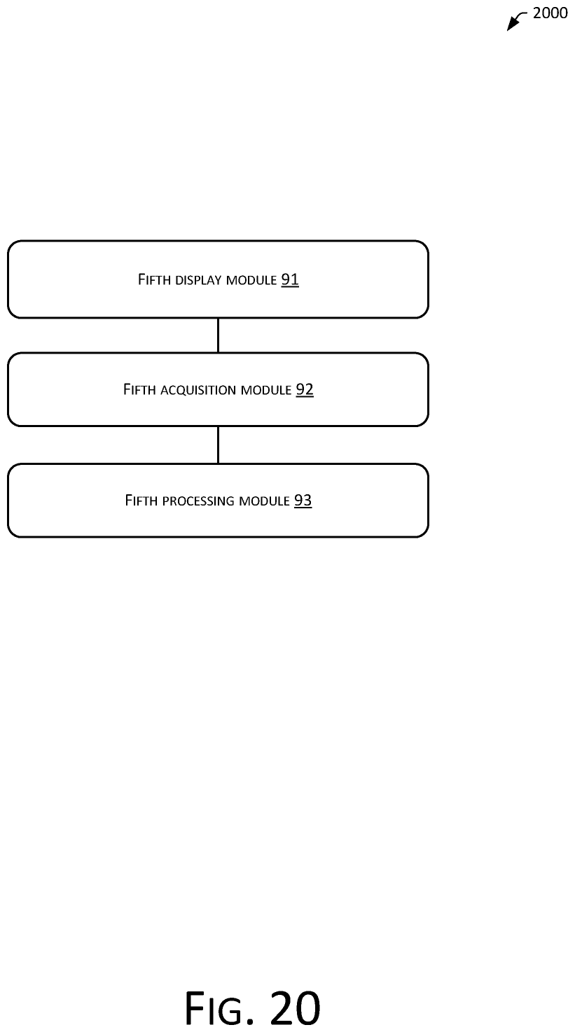

FIG. 20 is a schematic structural diagram of another genome feature extraction apparatus according to the embodiments of the present disclosure. Referring to FIG. 20, this embodiment provides another genome feature extraction apparatus 2000, which is capable of performing the genome feature extraction method corresponding to FIG. 19. Specifically, the feature extraction apparatus 2000 may include:

a fifth display module 2001 configured to display an interactive interface for implementing a feature extraction operation of a genome;

a fifth acquisition module 2002 configured to obtain a gene segment to be processed in response to an execution operation inputted on the interactive interface, the gene segment includes a base quality;

a fifth processing module 2003 configured to determine gene features of the gene segment and a data acquisition quality corresponding to the gene features, wherein the gene features are obtained by extraction based on a confidence level of the gene segment, and the confidence level of the gene segment is related to the base quality; and

the fifth display module 2001 further configured to display the gene features and the data acquisition quality in the interactive interface.

The apparatus shown in FIG. 20 can execute the method of the embodiments shown in FIG. 19. The parts of this embodiment that are not described in details can be referenced to related descriptions of the embodiments shown in FIG. 9. The implementation process and technical effect of the technical solution refer to the descriptions in the embodiments shown in FIG. 19, and are not repeatedly described herein.

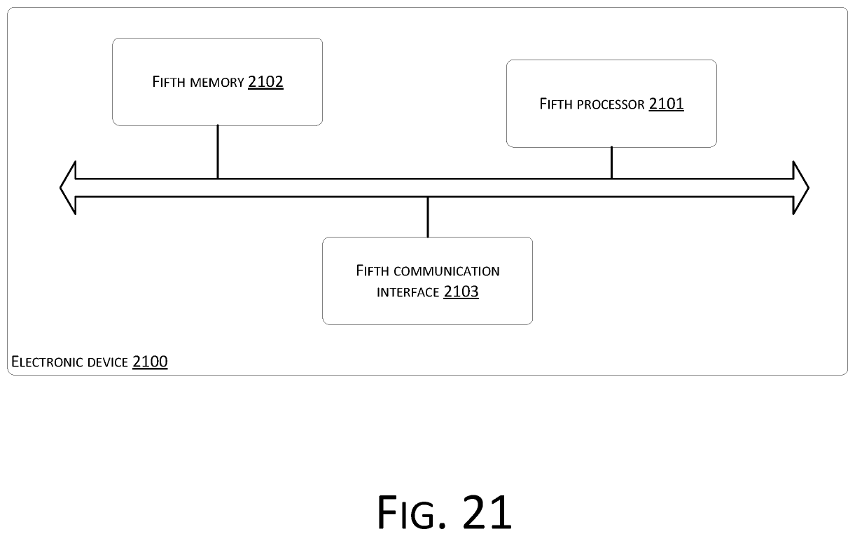

In a possible design, the structure of the genome feature extraction apparatus shown in FIG. 20 may be implemented as an electronic device. The electronic device may be a variety of devices, such as a mobile phone, a tablet computer, a server, etc. As shown in FIG. 21, an electronic device 2100 may include: a fifth processor 2101 and a fifth memory 2102. The fifth memory 2102 is configured to store a corresponding program of the electronic device for executing the genome feature extraction method provided in the embodiments shown in FIG. 19, and the fifth processor 2101 is configured to execute the program stored in the fifth memory 2102.

The program includes one or more computer instructions, wherein the one or more computer instructions, when executed by the fifth processor 2101, are capable of performing the following steps:

displaying an interactive interface for realizing a feature extraction operation of a genome;

obtaining a gene segment to be processed in response to an execution operation inputted on the interactive interface, the gene segment including a base quality;

determining gene features of the gene segment and a data acquisition quality corresponding to the gene features, wherein the gene features are obtained by extraction based on a confidence level of the gene segment, and the confidence level of the gene segments is related to the base quality; and

displaying the gene features and the data acquisition quality in the interactive interface.

Furthermore, the fifth processor 2101 is also configured to perform all or part of the steps in the embodiment shown in FIG. 19.

The electronic device 2100 may further include a fifth communication interface 2103 used for enabling the electronic device to communicate with other devices or a communication network.

In addition, the embodiments of the present disclosure provide a computer storage medium configured to store computer software instructions for an electronic device, which includes a program for executing the genome feature extraction method in the method embodiments shown in FIG. 19.

The foregoing apparatus embodiments are merely illustrative. Units described as separate parts may or may not be physically separate. Parts displayed as units may or may not be physical units, may be located in one place, or may be distributed over a plurality of network units. Some or all of the modules may be selected according to actual needs to achieve the purpose of the solutions of this embodiment. One of ordinary skill in the art can understand and implement them without making any inventive effort.

Through the above description of the embodiments, one skilled in the art will clearly understand that each embodiment can be implemented by adding a necessary general hardware platform, and apparently can also be implemented by a combination of hardware and software. With this understanding in mind, the essence of the above technical solutions or the portions that contribute to the existing technologies may be embodied in a form of a computer product. The present disclosure may adopt a form of a computer program product implemented on one or more computer-usable storage media (which includes, but are not limited to, disk storage, CD-ROM, optical storage, and the like) having computer-usable program codes embodied therein.

The present disclosure is described with reference to flowcharts and/or block diagrams of methods, apparatus (systems), and computer program products according to the embodiments of the present disclosure. It will be understood that each process and/or block of the flowcharts and/or block diagrams, and combinations of processes and/or blocks in the flowcharts and/or block diagrams, can be implemented by computer program instructions. These computer program instructions may be provided to a general purpose computer, a special purpose computer, an embedded processor, or a processor of other programmable device to produce a machine, to cause the instructions to generate an apparatus for implementing the functions specified in one or more processes of the flowcharts and/or one or more blocks of the block diagrams through the computer or the processor of other programmable device.

These computer program instructions may also be stored in a computer-readable storage device that can direct a computer or other programmable device to function in a particular manner, such that the instructions stored in the computer-readable storage device produce an article of manufacture including an instruction apparatus which implements the functions specified in one or more processes of the flowcharts and/or one or more blocks of the block diagrams.

These computer program instructions may also be loaded onto a computer or other programmable device to cause a series of operational steps to be performed on the computer or other programmable device so as to produce a computer implemented process, such that the instructions executed on the computer or other programmable device provide steps for implementing the functions specified in one or more processes of the flowcharts and/or one or more blocks of the block diagrams.

In a typical configuration, a computing device includes one or more processors (CPUs), input/output interface(s), network interface(s), and memory.

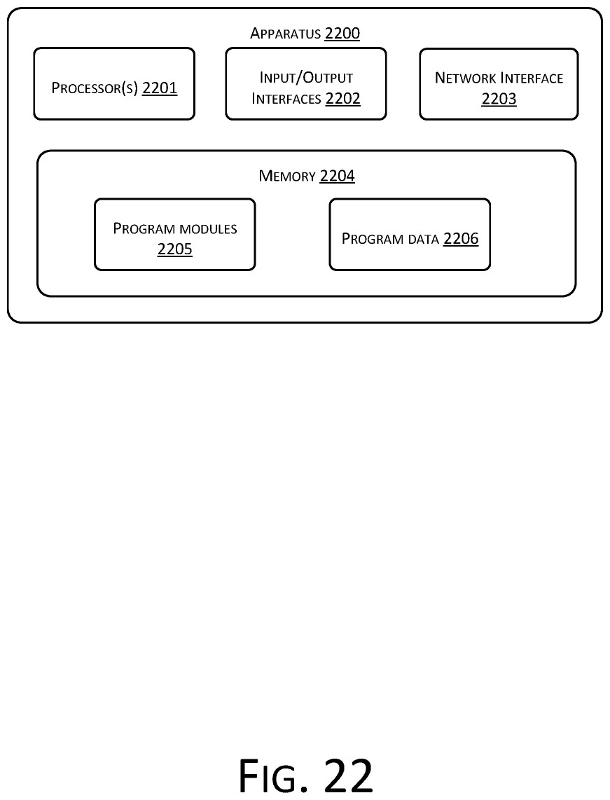

FIG. 22 shows an example apparatus 2200, such as the apparatuses shown in FIGS. 11, 13, 15, 17, and 20 in further details. In implementations, the example apparatus 2200 may represent any one of the foregoing apparatuses. By way of example and not limitation, the apparatus 2200 may include one or more processors 2201, an input/output (I/O) interface 2202, a network interface 2203, and memory 2204.

The memory 2204 may include a form of computer readable media such as a volatile memory, a random access memory (RAM) and/or a non-volatile memory, for example, a read-only memory (ROM) or a flash RAM. The memory 2204 is an example of a computer readable media. In implementations, the memory 2204 may include program units/modules 2205 and program data 2206. The program units/modules 2205 may include one or more of the foregoing units and/or modules as described in the foregoing embodiments and shown in the figures.

The memory 2204 may include a form of computer readable media such as a volatile memory, a random access memory (RAM) and/or a non-volatile memory, for example, a read-only memory (ROM) or a flash RAM. The memory 2204 is an example of a computer readable media.

The computer readable media may include a volatile or non-volatile type, a removable or non-removable media, which may achieve storage of information using any method or technology. The information may include a computer readable instruction, a data structure, a program module or other data. Examples of computer storage media include, but not limited to, phase-change memory (PRAM), static random access memory (SRAM), dynamic random access memory (DRAM), other types of random-access memory (RAM), read-only memory (ROM), electronically erasable programmable read-only memory (EEPROM), quick flash memory or other internal storage technology, compact disk read-only memory (CD-ROM), digital versatile disc (DVD) or other optical storage, magnetic cassette tape, magnetic disk storage or other magnetic storage devices, or any other non-transmission media, which may be used to store information that may be accessed by a computing device. As defined herein, the computer readable media does not include transitory media, such as modulated data signals and carrier waves.

Finally, it needs to be noted that: the above embodiments are only intended to illustrate the technical solutions of the present disclosure, but not to impose limitations thereon. Although the present disclosure has been described in detail with reference to the foregoing embodiments, one of ordinary skill in the art should understand that: the technical solutions described in the foregoing embodiments may be modified, or some technical features may be equivalently replaced. Such modifications or replacements do not depart from the spirit and scope of the corresponding technical solutions of the embodiments of the present disclosure.

The present disclosure can further be understood using the following clauses.

Clause 1: A method implemented by one or more computing devices, the method comprising: obtaining a gene segment to be processed, the gene segment including a base quality; determining a confidence level corresponding to the gene segment based on the base quality; and performing a feature extraction operation on the gene segment based on the confidence level corresponding to the gene segment to obtain gene features of the gene segment.

Clause 2: The method of Clause 1, wherein obtaining the gene segment to be processed comprises: obtaining a reference sequence and a gene sequence, wherein the gene sequence comprises a plurality of initial gene segments; and matching the reference sequence and the gene sequence to determine the gene segment to be processed from among the plurality of initial gene segments, wherein bases which do not match with the reference sequence exist in the gene segments, and a proportion of unmatched bases among the gene segments is larger than a preset threshold.

Clause 3: The method of Clause 1, wherein determining the confidence level corresponding to the gene segment based on the base quality comprises: obtaining ratio information between the base quality and 10; and determining the confidence level corresponding to the gene segment based on the ratio information, wherein the confidence level is positively correlated with the base quality and is less than 1.

Clause 4: The method of Clause 1, wherein performing the feature extraction operation on the gene segment based on the confidence level corresponding to the gene segment to obtain the gene features of the gene segment comprises: performing the feature extraction operation on the gene segment in a statistical counting mode based on the confidence level corresponding to the gene segment to obtain the gene features of the gene segment, wherein the gene features comprise: base information, base positions, and statistics corresponding to the base information.

Clause 5: The method of Clause 1, wherein after obtaining the gene features of the gene segment, the method further comprises: performing mutation detection on the gene segment based on the gene features to obtain a mutation detection result.

Clause 6: The method of Clause 5, wherein performing mutation detection on the gene segment based on the gene features to obtain the mutation detection result comprises: inputting the gene features into a three-dimensional network model to obtain the mutation detection result, wherein the three-dimensional network model is trained for performing mutation detections on gene segments based on respective gene features.

Clause 7: The method of Clause 5, wherein performing mutation detection on the gene segment based on the gene features comprises: obtaining mutation reference information corresponding to the gene segment based on the gene features, wherein the mutation reference information comprises at least one of the following information: 21-genotype prediction information, zygotic prediction information, first allelic mutation length information, and second allelic mutation length information; and obtaining a mutation detection result according to the mutation reference information.

Clause 8: The method of Clause 5, wherein after obtaining the mutation detection result, the method further comprises: performing disease prediction based on the mutation detection result.

Clause 9: A disease prediction method comprising: obtaining a disease prediction request, the disease prediction request comprising a gene segment to be predicted; determining gene features corresponding to the gene segment included in the disease prediction request, the gene features being extracted based on a confidence level of the gene segment, wherein the confidence level of the gene segment is related to a base quality included in the gene segment; and performing disease prediction on the gene segment based on the gene features to obtain a disease prediction result.

Clause 10: A genome feature extraction method comprising: determining a processing resource corresponding to a feature extraction service of a genome in response to a feature extraction request for calling the genome; performing the following steps with the processing resource: obtaining a gene segment to be processed, wherein the gene segment comprises a base quality; determining a confidence level corresponding to the gene segment based on the base quality; and performing a feature extraction operation on the gene segment based on the confidence level corresponding to the gene segment to obtain gene features of the gene segment.

Clause 11: A disease prediction method comprising: determining a processing resource corresponding to a disease prediction service in response to a call for a disease prediction request; and performing the following steps with the processing resource: obtaining the disease prediction request, wherein the disease prediction request comprises a gene segment to be predicted; determining gene features corresponding to the gene segment included in the disease prediction request, the gene features being extracted based on a confidence level of the gene segment, wherein the confidence level of the gene segment is related to a base quality included in the gene segment; and performing disease prediction on the gene segment based on the gene features to obtain a disease prediction result.

Clause 12: A genome feature extraction method comprising: displaying an interactive interface for realizing a feature extraction operation of a genome; obtaining a gene segment to be processed in response to an execution operation inputted on the interactive interface, wherein the gene segment comprises a base quality; determining gene features of the gene segment and a data acquisition quality corresponding to the gene features, wherein the gene features are obtained by extraction based on a confidence level of the gene segment, and the confidence level of the gene segment is related to the base quality; and displaying the gene features and the data acquisition quality in the interactive interface.

What is claimed is:

1. A method implemented by one or more computing devices, the method comprising:
obtaining a gene segment to be processed, the gene segment including a base quality;
determining a confidence level corresponding to the gene segment based on the base quality; and
performing a feature extraction operation on the gene segment based on the confidence level corresponding to the gene segment to obtain gene features of the gene segment.

2. The method of claim 1, wherein obtaining the gene segment to be processed comprises:
obtaining a reference sequence and a gene sequence, wherein the gene sequence comprises a plurality of initial gene segments; and
matching the reference sequence and the gene sequence to determine the gene segment to be processed from among the plurality of initial gene segments, wherein bases which do not match with the reference sequence exist in the gene segments, and a proportion of unmatched bases among the gene segments is larger than a preset threshold.

3. The method of claim 1, wherein determining the confidence level corresponding to the gene segment based on the base quality comprises:
obtaining ratio information between the base quality and 10; and
determining the confidence level corresponding to the gene segment based on the ratio information, wherein the confidence level is positively correlated with the base quality and is less than 1.

4. The method of claim 1, wherein performing the feature extraction operation on the gene segment based on the confidence level corresponding to the gene segment to obtain the gene features of the gene segment comprises:
performing the feature extraction operation on the gene segment in a statistical counting mode based on the confidence level corresponding to the gene segment to obtain the gene features of the gene segment, wherein the gene features comprise: base information, base positions, and statistics corresponding to the base information.

5. The method of claim 1, wherein after obtaining the gene features of the gene segment, the method further comprises:
performing mutation detection on the gene segment based on the gene features to obtain a mutation detection result.

6. The method of claim 5, wherein performing mutation detection on the gene segment based on the gene features to obtain the mutation detection result comprises:
inputting the gene features into a three-dimensional network model to obtain the mutation detection result, wherein the three-dimensional network model is trained for performing mutation detections on gene segments based on respective gene features.

7. The method of claim 5, wherein performing mutation detection on the gene segment based on the gene features comprises:
obtaining mutation reference information corresponding to the gene segment based on the gene features, wherein the mutation reference information comprises at least one of the following information: 21-genotype prediction information, zygotic prediction information, first allelic mutation length information, and second allelic mutation length information; and
obtaining a mutation detection result according to the mutation reference information.

8. The method of claim 5, wherein after obtaining the mutation detection result, the method further comprises:
performing disease prediction based on the mutation detection result.

9. One or more computer readable media storing executable instructions that, when executed by one or more processors, cause the one or more processors to perform acts comprising:
obtaining a gene segment to be processed, the gene segment including a base quality;
determining a confidence level corresponding to the gene segment based on the base quality; and
performing a feature extraction operation on the gene segment based on the confidence level corresponding to the gene segment to obtain gene features of the gene segment.

10. The one or more computer readable media of claim 9, wherein obtaining the gene segment to be processed comprises:
obtaining a reference sequence and a gene sequence, wherein the gene sequence comprises a plurality of initial gene segments; and
matching the reference sequence and the gene sequence to determine the gene segment to be processed from among the plurality of initial gene segments, wherein bases which do not match with the reference sequence exist in the gene segments, and a proportion of unmatched bases among the gene segments is larger than a preset threshold.

11. The one or more computer readable media of claim 9, wherein determining the confidence level corresponding to the gene segment based on the base quality comprises:
obtaining ratio information between the base quality and 10; and
determining the confidence level corresponding to the gene segment based on the ratio information, wherein the confidence level is positively correlated with the base quality and is less than 1.

12. The one or more computer readable media of claim 9, wherein performing the feature extraction operation on the gene segment based on the confidence level corresponding to the gene segment to obtain the gene features of the gene segment comprises:
performing the feature extraction operation on the gene segment in a statistical counting mode based on the confidence level corresponding to the gene segment to obtain the gene features of the gene segment, wherein the gene features comprise: base information, base positions, and statistics corresponding to the base information.

13. The one or more computer readable media of claim 9, wherein after obtaining the gene features of the gene segment, the acts further comprise:
performing mutation detection on the gene segment based on the gene features to obtain a mutation detection result.

14. The one or more computer readable media of claim 13, wherein performing mutation detection on the gene segment based on the gene features to obtain the mutation detection result comprises:
inputting the gene features into a three-dimensional network model to obtain the mutation detection result, wherein the three-dimensional network model is trained for performing mutation detections on gene segments based on respective gene features.

15. The one or more computer readable media of claim 13, wherein performing mutation detection on the gene segment based on the gene features comprises:
obtaining mutation reference information corresponding to the gene segment based on the gene features, wherein the mutation reference information comprises at least one of the following information: 21-genotype prediction information, zygotic prediction information, first allelic mutation length information, and second allelic mutation length information; and
obtaining a mutation detection result according to the mutation reference information.

16. The one or more computer readable media of claim 13, wherein after obtaining the mutation detection result, the acts further comprise:
performing disease prediction based on the mutation detection result.

17. An apparatus comprising:
one or more processors;
memory storing executable instructions that, when executed by the one or more processors, cause the one or more processors to perform acts comprising:
obtaining a gene segment to be processed, the gene segment including a base quality;
determining a confidence level corresponding to the gene segment based on the base quality; and
performing a feature extraction operation on the gene segment based on the confidence level corresponding to the gene segment to obtain gene features of the gene segment.

18. The apparatus of claim 17, wherein obtaining the gene segment to be processed comprises:
obtaining a reference sequence and a gene sequence, wherein the gene sequence comprises a plurality of initial gene segments; and
matching the reference sequence and the gene sequence to determine the gene segment to be processed from among the plurality of initial gene segments, wherein bases which do not match with the reference sequence exist in the gene segments, and a proportion of unmatched bases among the gene segments is larger than a preset threshold.

19. The apparatus of claim 17, wherein determining the confidence level corresponding to the gene segment based on the base quality comprises:
obtaining ratio information between the base quality and 10; and
determining the confidence level corresponding to the gene segment based on the ratio information, wherein the confidence level is positively correlated with the base quality and is less than 1.

20. The apparatus of claim 17, wherein performing the feature extraction operation on the gene segment based on the confidence level corresponding to the gene segment to obtain the gene features of the gene segment comprises:
performing the feature extraction operation on the gene segment in a statistical counting mode based on the confidence level corresponding to the gene segment to obtain the gene features of the gene segment, wherein the gene features comprise: base information, base positions, and statistics corresponding to the base information.
